(19) United States
(12) Patent Application Publication
(10) Pub. No.: US 2024/0041860 A1
(43) Pub. Date: Feb. 8, 2024

(54) RESTORATION OF MOTOR FUNCTION POST-NEUROLOGICAL INJURY USING PSYCHEDELICS

(71) Applicant: THE JOHNS HOPKINS UNIVERSITY, Baltimore, MD (US)

(72) Inventors: Gul Dolen, Baltimore, MD (US); John Krakauer, Baltimore, MD (US); Romain Nardou, Baltimore, MD (US); Steven Robert Zeiler, Baltimore, MD (US); Junia L. De Deus, Baltimore, MD (US)

(21) Appl. No.: 18/255,518

(22) PCT Filed: Dec. 6, 2021
(86) PCT No.: PCT/US21/62074

§ 371 Date: Jun. 1, 2023

Related U.S. Application Data
(60) Provisional application No. 63/120,657, filed on Dec. 2, 2020.

(51) Int. Cl. A61K 31/48; A61K 31/4045; A61K 31/675; A61K 31/36; A61K 31/135; A61P 25/28; A61H 3/00

(52) U.S. Cl. CPC A61K 31/48; A61K 31/4045; A61K 31/675; A61K 31/36; A61K 31/135; A61P 25/28; A61H 3/00

(57) ABSTRACT

Methods of improving motor function in subjects diagnosed with a central nervous system injury are contemplated as including an administration of a psychedelic, empathogenic, or dissociative compound in a pharmaceutically effective amount followed by administration of a motor skill restoration therapy. The psychedelic, empathogenic, or dissociative compound may be administered to a subject that has been determined to be therapy refractive with a chronic central nervous system injury, to a subject with a subacute central nervous system injury sustained within 7 days and 6 months prior, or to a subject with an acute central nervous system injury sustained within the past 7 days. Such methods may be seen improve motor function in the subjects.

This application claims priority to U.S. provisional patent application Ser. No. 63/120,657, filed Dec. 2, 2020, which is incorporated herein by reference in its entirety.

### BACKGROUND OF THE INVENTION

The invention relates to the field of methods for the use of psychedelic, empathogenic, or dissociative compounds for regaining motor function following an injury to the central nervous system. After a subject sustains an injury to the central nervous system it has been shown that substantial spontaneous recovery occurs in the weeks and/or months after the injury takes place. This period is known as the critical window for relearning motor skills, during which the plasticity of the brain is increased such that neuronal circuits have increased responsiveness to training and experience, mimicking conditions during development (Chen et al. PM R. 2010; 2(12 Suppl 2):S306-S312). The increased plasticity associated with the critical window allows the subject to restore, at least to a degree, motor skills that were impaired as a result of the central nervous system injury. However, a critical window eventually closes. Once closed, the neuroplasticity of the brain decreases, resulting in little to no improvement in motor function, despite continued administration of motor training. Thus, there is an urgent need for new approaches for reopening the critical window following central nervous system injury.

### SUMMARY OF THE INVENTION

The invention provides methods for improving motor function in subjects with central nervous system injury (e.g., an acute central nervous system injury, a subacute central nervous system injury, or a chronic central nervous system injury) and for treating focal diaschisis caused by a central nervous system injury in a subject in need thereof.

The invention further provides compositions for use in improving in treating a central nervous system injury or focal diastasis.

In a first aspect, the disclosure provides a method of improving motor function in a therapy-refractory subject diagnosed with a central nervous system injury (e.g., a chronic central nervous system injury or a subacute central nervous system injury), the method including: (i) administering to the subject a pharmaceutical composition comprising a psychedelic compound at least 3 months after sustaining the central nervous system injury, and (ii) after step (i), administering a motor skill restoration therapy, thereby improving motor function in the therapy-refractory subject. After the initial administration of the psychedelic compound, the subject may be administered the psychedelic compound while the subject is also being administered a motor skill restoration therapy. For example, after the initial administration of the psychedelic compound, the subject may already be being administered the motor skill restoration therapy when the subject is administered the psychedelic compound for a second time.

In another aspect, the disclosure provides a method of improving motor function in a subject diagnosed with a central nervous system injury (e.g., an acute central nervous system injury or a subacute central nervous system injury), the method including: (i) administering to the subject a pharmaceutical composition including a psychedelic compound within 3 months (e.g., between 1 week and 12 weeks, 1 week and 10 weeks, 1 week and 8 weeks, 1 week and 6 weeks, 1 week and 4 weeks, 1 week and 2 weeks, 2 weeks and 10 weeks, 4 weeks and 8 weeks, 6 weeks and 12 weeks, 1 day and 5 days, 5 days and 10 days, 10 days and 90 days, 15 days and 80 days, 20 days and 60 days, 25 days and 40 days, 1 day and 90 days, and 40 days and 90 days) of sustaining the central nervous system injury (e.g., the acute central nervous system injury or the subacute central nervous system injury), and (ii) after step (i), administering a motor skill restoration therapy, thereby improving motor function in the subject.

In another aspect the disclosure provides a method of treating focal diaschisis caused by a central nervous system injury in a subject, the method including: (i) administering to the subject a pharmaceutical composition comprising a psychedelic compound, and (ii) after step (i), administering a motor skill restoration therapy, thereby improving motor function in the subject. In some embodiments, the subject suffers from crossed cerebellar diaschisis (CCD). In certain embodiments, the subject suffers from stroke, concussion, or chronic traumatic encephalopathy (CTE).

In certain embodiments, the motor skill restoration therapy is administered for an additional 2 to 4 weeks (e.g., 14 days to 28 days, 14 days to 21 days, 14 days to 18 days, 18 days to 21 days, and 21 days to 28 days) after the psychedelic compound is no longer being administered to the subject. In some embodiments, the motor skill restoration therapy is administered within 24 hours (e.g. within 1 hour, 5 hours, 10 hours, 15 hours, 20 hours, and 24 hours) of the psychedelic compound. In certain embodiments, the motor skill restoration therapy is administered within 6 hours (e.g., within 1 hour, 2 hours, 3 hours, 4 hours, 5 hours, and 6 hours) of the psychedelic compound. In particular embodiments, the motor skill restoration therapy is administered within 1 hour (e.g., 10 minutes, 20 minutes, 30 minutes, 40 minutes, 50 minutes, and 60 minutes) of the psychedelic compound.

In some embodiments, the motor skill restoration therapy comprises a rehabilitation program and/or a therapeutic compound. In particular embodiments, the motor skill restoration therapy includes a rehabilitation program. In certain embodiments, the rehabilitation program includes physical therapy, occupational therapy, or speech therapy. In some embodiments, the rehabilitation program comprises a robot assisted therapy. In particular embodiments, the robot-assisted therapy is an end-effector type robotic device therapy. In certain embodiments, the robot-assisted therapy is an exoskeleton-type robotic device therapy. In certain embodiments, the rehabilitation program utilizes an approach known as the MindPod Dolphin. The MindPod is an immersive therapy platform to help motivate patients to train complex upper body exploratory movements for motor recovery; when required, the MindPod will be paired with exoskeletal support. In some cases, alternative technologies to be the MindPod Dolphin may be used. In some embodiments, the rehabilitation program comprises a 3D virtual reality video game “Bandit's Shark Showdown. In some embodiment, the rehabilitation program comprises deep brain stimulation.

In some embodiments, the rehabilitation program comprises interventions with technologies designed to enhance the intensity of the post-injury rehabilitation.

In some embodiments, the central nervous system injury results from a stroke (e.g., ischemic or hemorrhagic). In some embodiments, the central nervous system injury is a traumatic brain injury. In certain embodiments, the central nervous system injury is a spinal cord injury (e.g. a spinal cord injury caused by a tumor or multiple sclerosis.

In particular embodiments, the subject is administered an alpha adrenergic drug (e.g., clonidine).

In some embodiments, the motor function is a fine or a gross motor function. In certain embodiments, the motor function is a fine motor function. In particular embodiments, the fine motor function is speech

In certain embodiments, the psychedelic compound is administered every 3 to 4 days. In some embodiments, the psychedelic compound is administered every 1 to 2 weeks. In some embodiments, the psychedelic compound is administered every 3 to 4 weeks.

In some embodiments, the psychedelic compound is a 5-HT_{2A} receptor agonist. In particular embodiments, the 5-HT_{2A} receptor agonist is lysergic acid diethylamide (LSD). In some embodiments, said pharmaceutical composition is a unit dosage form including from 5 to 250 μg (e.g., 10 μg, 25 μg, 50 μg, 100 μg, 125 μg, 150 μg, 175 μg, 200 μg, 225 μg, and 250 μg) of lysergic acid diethylamide or a pharmaceutically acceptable salt thereof. In particular embodiments, the unit dosage is an oral dose. In some embodiments, the unit dosage form includes from 50 to 250 μg (e.g., 50±10 μg, 60±10 μg, 70±10 μg, 80±10 μg, 90±10 μg, 100±10 μg, 110±10 μg, 120±10 μg, 130±10 μg, 140±10 μg, 150±10 μg, 160±10 μg, 170±10 μg, 180±10 μg, 190±10 μg, 200±10 μg, 210±10 μg, 220±10 μg, 230±10 μg, 240±10 μg, and 250±10 μg) of LSD or a pharmaceutically acceptable salt thereof. In certain embodiments, the unit dosage form includes from 5 to 30 μg (e.g., 5±5 μg, 10±5 μg, 15±5 μg, 20±5 μg, 25±5 μg, and 30±5 μg) of LSD or a pharmaceutically acceptable salt thereof. In particular embodiments, said pharmaceutical composition is a unit dosage form including 10±5 μg of LSD or a pharmaceutically acceptable salt thereof.

In some embodiments, the 5-HT_{2A} receptor agonist is psilocybin or psilocin. In certain embodiments, said pharmaceutical composition is a unit dosage form including from 1 to 40 mg (e.g., 2±1 mg, 3±1 mg, 4±1 mg, 5±1 mg, 6±1 mg, 7±1 mg, 8±1 mg, 9±1 mg, 10±5 mg, 15±5 mg, 20±5 mg, 25±5 mg, 30±5 mg, 35±5 mg, and 40±5 mg)) of psilocybin or psilocin, or a pharmaceutically acceptable salt thereof. In certain embodiments, the unit dosage is an oral dose. In some embodiments, the unit dosage form includes from 1 to 10 mg (e.g., 2±1 mg, 3±1 mg, 4±1 mg, 5±1 mg, 6±1 mg, 7±1 mg, 8±1 mg, 9±1 mg, and 10±1 mg) of psilocybin or psilocin, or a pharmaceutically acceptable salt thereof. In particular embodiments, the unit dosage form includes from 5 to 40 mg (e.g., 5±5 mg, 10±5 mg, 15±5 mg, 20±5 mg, 25±5 mg, 30±5 mg, 35±5 mg, and 40±5 mg) of psilocybin or psilocin, or a pharmaceutically acceptable salt thereof.

In some embodiments, the psychedelic compound or pharmaceutically acceptable salt thereof is administered orally, intravenously, by inhalation, by nebulization, by aerosolization, intranasally, intratracheally, intrabronchially, subcutaneously, or intramuscularly. In certain embodiments, the psychedelic compound or pharmaceutically acceptable salt thereof is administered intravenously. In particular embodiments, the psychedelic compound or pharmaceutically acceptable salt thereof is administered orally.

In another aspect, the disclosure provides a method of improving motor function in a therapy-refractory subject diagnosed with a central nervous system injury (e.g., a chronic central nervous system injury or a subacute central nervous system injury), the method including: (i) administering to the subject a pharmaceutical composition comprising an empathogenic compound at least 3 months after sustaining the central nervous system injury, and (ii) after step (i), administering a motor skill restoration therapy, thereby improving motor function in the therapy-refractory subject. After the initial administration of the psychedelic compound, the subject may be administered the psychedelic compound while the subject is also being administered a motor skill restoration therapy. For example, after the initial administration the empathogenic compound, the subject may already be being administered the motor skill restoration therapy when the subject is administered the empathogenic compound for a second time.

In another aspect the disclosure provides a method of improving motor function in a subject diagnosed with a central nervous system injury (e.g., an acute central nervous system injury or a subacute central nervous system injury), the method including: (i) administering to the subject a pharmaceutical composition comprising an empathogenic compound within 3 months (e.g., between 1 week and 12 weeks, 1 week and 10 weeks, 1 week and 8 weeks, 1 week and 6 weeks, 1 week and 4 weeks, 1 week and 2 weeks, 2 weeks and 10 weeks, 4 weeks and 8 weeks, 6 weeks and 12 weeks, 1 day and 5 days, 5 days and 10 days, 10 days and 90 days, 15 days and 80 days, 20 days and 60 days, 25 days and 40 days, 1 day and 90 days, and 40 days and 90 days) of sustaining the central nervous system injury (e.g., the acute central nervous system injury or the subacute central nervous system injury), and (ii) after step (i), administering a motor skill restoration therapy, thereby improving motor function in the subject.

In another aspect the disclosure provides a method of treating focal diaschisis caused by a central nervous system injury in a subject, the method including: (i) administering to the subject a pharmaceutical composition including an empathogenic compound, and (ii) after step (i), administering a motor skill restoration therapy, thereby improving motor function in the subject. In some embodiments, the subject suffers from CCD. In certain embodiments, the subject suffers from stroke, concussion, or CTE.

In certain embodiments, the motor skill restoration therapy is administered for an additional 2 to 4 weeks (e.g., 14 days to 28 days, 14 days to 21 days, 14 days to 18 days, 18 days to 21 days, and 21 days to 28 days) after the empathogenic compound is no longer being administered to the subject. In some embodiments, the motor skill restoration therapy is administered within 24 hours (e.g. within 1 hour, 5 hours, 10 hours, 15 hours, 20 hours, and 24 hours) of the empathogenic compound. In certain embodiments, the motor skill restoration therapy is administered within 6 hours (e.g., within 1 hour, 2 hours, 3 hours, 4 hours, 5 hours, and 6 hours) of the empathogenic compound. In particular embodiments, the motor skill restoration therapy is administered within 1 hour (e.g., 10 minutes, 20 minutes, 30 minutes, 40 minutes, 50 minutes, and 60 minutes) of the empathogenic compound.

In some embodiments, the motor skill restoration therapy includes a rehabilitation program and/or a therapeutic compound. In particular embodiments, the motor skill restoration therapy includes a rehabilitation program. In certain embodiments, the rehabilitation program includes physical therapy, occupational therapy, or speech therapy.

In some embodiments, the rehabilitation program comprises a robot assisted therapy. In particular embodiments, the robot assisted therapy is an end-effector type robotic device therapy. In certain embodiments, the robot assisted therapy is an exoskeleton-type robotic device therapy. In certain embodiments, the rehabilitation utilizes an approach known as the MindPod Dolphin. The MindPod is an immersive therapy platform to help motivate patients to train complex upper body exploratory movements for motor recovery; when required, the MindPod will be paired with exoskeletal support. In some cases, alternative technologies to be the MindPod Dolphin may be used. In some embodiments, the rehabilitation program comprises a 3D virtual reality video game “Bandit's Shark Showdown. In some embodiment, the rehabilitation program comprises deep brain stimulation.

In some embodiments, the rehabilitation program comprises interventions with technologies designed to enhance the intensity of the post-injury rehabilitation.

In some embodiments, the central nervous system injury results from a stroke (e.g. hemorrhagic or ischemic). In some embodiments, the central nervous system injury is a traumatic brain injury. In certain embodiments, the central nervous system injury is a spinal cord injury (e.g. a spinal cord injury caused by a tumor or multiple sclerosis).

In particular embodiments, the subject is administered an alpha adrenergic drug (e.g., clonidine).

In some embodiments, the motor function is a fine or a gross motor function. In certain embodiments, the motor function is a fine motor function. In particular embodiments, the fine motor function is speech

In certain embodiments, the empathogenic compound is administered every 3 to 4 days. In some embodiments, the empathogenic compound is administered every 1 to 2 weeks. In some embodiments, the empathogenic compound is administered every 3 to 4 weeks.

In some embodiments, the empathogenic compound is 3,4-methylenedioxymethamphetamine (MDMA). In some embodiments, said pharmaceutical composition is a unit dosage form including from 50 to 150 mg (e.g., 55 mg, 60 mg, 70 mg, 80 mg, 90 mg, 100 mg, 110 mg, 120 mg, 130 mg, 140 mg, and 150 mg) of MDMA or a pharmaceutically acceptable salt thereof. In particular embodiments, the unit dosage is an oral dose. In some embodiments, the unit dosage form includes from 50 to 75 mg (e.g., 50±5 mg, 55±5 mg, 60±mg, 65±5 mg, 70±5 mg, and 75±5 mg) of MDMA or a pharmaceutically acceptable salt thereof. In certain embodiments, the unit dosage form includes from 75 to 150 mg (e.g., 75±5 mg, 80±5 mg, 85±5 mg, 90±5 mg, 95±5 mg, 100±5 mg, 105±5 mg, 110±5 mg, 115±5 mg, 120±5 mg, 125±5 mg, 130±5 mg, 135±5 mg, 140±5 mg, 145±5 mg, and 150±5 mg) of MDMA or a pharmaceutically acceptable salt thereof. In particular embodiments, the unit dosage form includes 75 mg of MDMA or a pharmaceutically acceptable salt thereof.

In some embodiments, the empathogenic compound or pharmaceutically acceptable salt thereof is administered orally, intravenously, by inhalation, by nebulization, by aerosolization, intranasally, intratracheally, intrabronchially, subcutaneously, or intramuscularly.

In another aspect, the disclosure provides a method of improving motor function in a therapy-refractory subject diagnosed with a central nervous system injury (e.g., a chronic central nervous system injury or a subacute central nervous system injury), the method including: (i) administering to the subject a pharmaceutical composition comprising a dissociative compound at least 3 months after sustaining the central nervous system injury, and (ii) after step (i), administering a motor skill restoration therapy, thereby improving motor function in the therapy-refractory subject. After the initial administration of the psychedelic compound, the subject may be administered the psychedelic compound while the subject is also being administered a motor skill restoration therapy. For example, after the initial administration the dissociative compound, the subject may already be being administered the motor skill restoration therapy when the subject is administered the dissociative compound for a second time.

In another aspect the disclosure provides a method of improving motor function in a subject diagnosed with a central nervous system injury (e.g., an acute central nervous system injury or a subacute central nervous system injury), the method including: (i) administering to the subject a pharmaceutical composition comprising a dissociative compound within 3 months (e.g., between 1 week and 12 weeks, 1 week and 10 weeks, 1 week and 8 weeks, 1 week and 6 weeks, 1 week and 4 weeks, 1 week and 2 weeks, 2 weeks and 10 weeks, 4 weeks and 8 weeks, 6 weeks and 12 weeks, 1 day and 5 days, 5 days and 10 days, 10 days and 90 days, 15 days and 80 days, 20 days and 60 days, 25 days and 40 days, 1 day and 90 days, and 40 days and 90 days) of sustaining the central nervous system injury (e.g., the acute central nervous system injury or the subacute central nervous system injury), and (ii) after step (i), administering a motor skill restoration therapy, thereby improving motor function in the subject.

In another aspect the disclosure provides a method of treating focal diaschisis caused by a central nervous system injury in a subject, the method including: (i) administering to the subject a pharmaceutical composition comprising a dissociative compound, and (ii) after step (i), administering a motor skill restoration therapy, thereby improving motor function in the subject. In some embodiments, the subject suffers from CCD. In certain embodiments, the subject suffers from stroke, concussion, or CTE.

In certain embodiments, the motor skill restoration therapy is administered for an additional 2 to 4 weeks (e.g., 14 days to 28 days, 14 days to 21 days, 14 days to 18 days, 18 days to 21 days, and 21 days to 28 days) after the dissociative compound is no longer being administered to the subject. In some embodiments, the motor skill restoration therapy is administered within 24 hours (e.g. within 1 hour, 5 hours, 10 hours, 15 hours, 20 hours, and 24 hours) of the dissociative compound. In certain embodiments, the motor skill restoration therapy is administered within 6 hours (e.g., within 1 hour, 2 hours, 3 hours, 4 hours, 5 hours, and 6 hours) of the dissociative compound. In particular embodiments, the motor skill restoration therapy is administered within 1 hour (e.g., 10 minutes, 20 minutes, 30 minutes, 40 minutes, 50 minutes, and 60 minutes) of the dissociative compound.

In some embodiments, the motor skill restoration therapy comprises a rehabilitation program and/or a therapeutic compound. In particular embodiments, the motor skill restoration therapy includes a rehabilitation program. In certain embodiments, the rehabilitation program includes physical therapy, occupational therapy, or speech therapy.

In some embodiments, the rehabilitation program comprises a robot assisted therapy. In particular embodiments, the robot assisted therapy is an end-effector type robotic device therapy. In certain embodiments, the robot assisted therapy is an exoskeleton-type robotic device therapy. In certain embodiments, the rehabilitation program an approach known as the MindPod Dolphin. The MindPod is an immersive therapy platform to help motivate patients to train complex upper body exploratory movements for motor recovery; when required, the MindPod will be paired with exoskeletal support. In some cases, alternative technologies to be the MindPod Dolphin may be used. In some embodiments, the rehabilitation program comprises a 3D virtual reality video game “Bandit's Shark Showdown. In some embodiment, the rehabilitation program comprises deep brain stimulation.

In some embodiments, the rehabilitation program comprises interventions with technologies designed to enhance the intensity of the post-injury rehabilitation.

In some embodiments, the central nervous system injury may be the result of a stroke (e.g. ischemic or hemorrhagic). In some embodiments, the central nervous system injury is a traumatic brain injury. In certain embodiments, the central nervous system injury is a spinal cord injury (e.g. a spinal cord injury caused by a tumor or multiple sclerosis).

In particular embodiments, the subject is administered an alpha adrenergic drug.

In some embodiments, the motor function is a fine or a gross motor function. In certain embodiments, the motor function is a fine motor function. In particular embodiments, the fine motor function is speech

In certain embodiments, the dissociative compound is administered every 3 to 4 days. In some embodiments, wherein the dissociative compound is administered every 1 to 2 weeks. In some embodiments, the dissociative compound is administered every 3 to 4 weeks.

In some embodiments, the dissociative compound is ketamine. In some embodiments, said pharmaceutical composition is a unit dosage form including from 50 to 200 mg (e.g., 50±10 mg, 60±10 mg, 70±10 mg, 80±10 mg, 90±10 mg, 100±10 mg, 110±10 mg, 120±10 mg, 130±10 mg, 140±10 mg, 150±10 mg, 160±10 mg, 170±10 mg, 180±10 mg, 190±10 mg, and 200±10 mg) of ketamine or a pharmaceutically acceptable salt thereof. In particular embodiments, the unit dosage is an intravenous dose. In some embodiments, the unit dosage form includes from 100±10 mg of ketamine or a pharmaceutically acceptable salt thereof.

In some embodiments, the dissociative compound or pharmaceutically acceptable salt thereof is administered orally, intravenously, by inhalation, by nebulization, by aerosolization, intranasally, intratracheally, intrabronchially, subcutaneously, or intramuscularly.

In some embodiments, the subject is human.

In another aspect, the disclosure provides a psychedelic compound for use in the treatment of a central nervous system injury, focal diaschisis or an injury associated with impaired motor function in a subject. Suitably, the central nervous system injury, focal diaschisis or an injury associated with impaired motor function may be acute, subacute, or chronic. Suitably, the focal diaschisis may be caused by a central nervous system injury in a subject. Suitably, the central nervous system injury may a traumatic brain injury or a spinal cord injury (e.g. a spinal cord injury caused by a tumor or multiple sclerosis) or may be caused by a stroke. Suitably, the subject may suffer from crossed cerebellar diaschisis (CCD). Suitably, the subject may suffer from stroke, concussion, or chronic traumatic encephalopathy (CTE). Suitably, the subject may be a therapy-refractory subject.

Suitably, the psychedelic compound may be administered within 24 hours (e.g. within 1 hour, 5 hours, 10 hours, 15 hours, 20 hours, and 24 hours) prior to a subject undergoing motor skill restoration therapy. Suitably, the psychedelic compound may be administered within 6 hours (e.g., within 1 hour, 2 hours, 3 hours, 4 hours, 5 hours, and 6 hours) prior to a subject undergoing motor skill restoration therapy. Suitably, the psychedelic compound may be administered within 1 hour (e.g., 10 minutes, 20 minutes, 30 minutes, 40 minutes, 50 minutes, and 60 minutes) prior to a subject undergoing motor skill restoration therapy. Suitably, the motor skill restoration therapy may be administered for an additional 2 to 4 weeks after the psychedelic compound is no longer being administered to the subject. Suitably, the motor skill restoration therapy may comprise a rehabilitation program and/or a therapeutic compound. Suitably, the motor skill restoration therapy may include a rehabilitation program such as physical therapy, occupational therapy, or speech therapy.

Suitably, the rehabilitation program may be a robot assisted therapy. Suitably, the robot assisted therapy may be an end-effector type robotic device therapy. Suitably, the robot assisted therapy may be an exoskeleton-type robotic device therapy. Suitably, the rehabilitation program may utilize an approach known as the MindPod Dolphin. Suitably, alternative technologies to be the MindPod Dolphin may be used. Suitably, the rehabilitation program may comprise a 3D virtual reality video game “Bandit's Shark Showdown. Suitably, the rehabilitation program may comprise deep brain stimulation.

Suitably, the rehabilitation program comprises interventions with technologies designed to enhance the intensity of the post-injury rehabilitation. Suitably, the central nervous system injury may be the results of a stroke (e.g. ischemic or hemorrhagic), brain injury or spinal cord injury (e.g. a spinal cord injury caused by a tumor or multiple sclerosis).

Suitably, the psychedelic compound may be for use in combination with an alpha adrenergic drug (e.g., clonidine) or an analog thereof.

Suitably, the central nervous system injury may impact motor function, such as fine or gross. motor function. Suitably, the motor function may be a fine motor function. Suitably, the fine motor function may be speech

Suitably, the psychedelic compound may be formulated for administration every 3 to 4 days. Suitably, the psychedelic compound may be formulated for administration every 1 to 2 weeks. Suitably, the psychedelic compound may be formulated for administration every 3 to 4 weeks.

Suitably, the psychedelic compound may be a 5-HT_{2A} receptor agonist. In particular embodiments, the 5-HT_{2A} receptor agonist is lysergic acid diethylamide (LSD). Suitably, the psychedelic compound may be a unit dosage form including from 5 to 250 μg (e.g., 50±10 μg, 60±10 μg, 70±10 μg, 80±10 μg, 90±10 μg, 100±10 μg, 110±10 μg, 120±10 μg, 130±10 μg, 140±10 μg, 150±10 μg, 160±10 μg, 170±10 μg, 180±10 μg, 190±10 μg, 200±10 μg, 210±10 μg, 220±10 μg, 230±10 μg, 240±10 μg, and 250±10 μg) of lysergic acid diethylamide or a pharmaceutically acceptable salt thereof. Suitably, the unit dosage may be an oral dose. Suitably, the unit dosage form of the psychedelic compound may comprise from 50 to 250 μg (e.g., 50±10 μg, 60±10 μg, 70±10 μg, 80±10 μg, 90±10 μg, 100±10 μg, 110±10 μg, 120±10 μg, 130±10 μg, 140±10 μg, 150±10 μg, 160±10 μg, 170±10 μg, 180±10 μg, 190±10 μg, 200±10 μg, 210±10 μg, 220±10 μg, 230±10 μg, 240±10 μg, and 250±10 μg) of LSD or a pharmaceutically acceptable salt thereof. Suitably, the unit dosage form of the psychedelic compound may comprise from 5 to 30 μg (e.g., 5±5 μg, 10±5 μg, 15±5 μg, 20±5 μg, 25±5 μg, and 30±5 μg) of LSD or a pharmaceutically acceptable salt thereof. Suitably, the unit dosage form of the psychedelic compound may comprise 10±5 μg of LSD or a pharmaceutically acceptable salt thereof.

Suitably, the 5-HT_{2A} receptor agonist may be psilocybin or psilocin. Suitably, the psychedelic compound may be a unit dosage form including from 1 to 40 mg (e.g., 2±1 mg, 3±1 mg, 4±1 mg, 5±1 mg, 6±1 mg, 7±1 mg, 8±1 mg, 9±1 mg, 10±5 mg, 15±5 mg, 20±5 mg, 25±5 mg, 30±5 mg, 35±5 mg, and 40±5 mg)) of psilocybin or psilocin, or a pharmaceutically acceptable salt thereof. Suitably, the psychedelic compound may be a unit dosage form including from 1 to 10 mg (e.g., 2±1 mg, 3±1 mg, 4±1 mg, 5±1 mg, 6±1 mg, 7±1 mg, 8±1 mg, 9±1 mg, and 10±1 mg) of psilocybin or psilocin, or a pharmaceutically acceptable salt thereof. Suitably the psychedelic compound may be a unit dosage form including from 5 to 40 mg (e.g., 5±5 mg, 10±5 mg, 15±5 mg, 20±5 mg, 25±5 mg, 30±5 mg, 35±5 mg, and 40±5 mg) of psilocybin or psilocin, or a pharmaceutically acceptable salt thereof.

Suitably, the psychedelic compound or pharmaceutically acceptable salt thereof may be administered orally, intravenously, by inhalation, by nebulization, by aerosolization, intranasally, intratracheally, intrabronchially, subcutaneously, or intramuscularly. Suitably, the psychedelic compound or pharmaceutically acceptable salt thereof may be administered intravenously. Suitably, the psychedelic compound or pharmaceutically acceptable salt thereof may be administered orally.

Suitably, the psychedelic compound may be comprised in a pharmaceutical composition. Accordingly, another aspect provides a pharmaceutical composition comprising a psychedelic compound disclosed herein for use in the treatment of a central nervous system injury or focal diaschisis in a subject.

In another aspect, there is provided an empathogenic compound for use in the treatment of a central nervous system injury, focal diaschisis or an injury associated with impaired motor function in a subject. Suitably, the central nervous system injury, focal diaschisis or an injury associated with impaired motor function may be acute, subacute, or chronic. Suitably, the focal diaschisis may be caused by a central nervous system injury in a subject. Suitably, the central nervous system injury may a traumatic brain injury or a spinal cord injury (e.g. a spinal cord injury caused by a tumor or multiple sclerosis) or may be caused by a stroke. Suitably, the subject may suffer from crossed cerebellar diaschisis (CCD). Suitably, the subject may suffer from stroke (e.g. ischemic or hemorrhagic), concussion, or chronic traumatic encephalopathy (CTE). Suitably, the subject may be a therapy-refractory subject.

Suitably, the empathogenic compound may be administered within 24 hours (e.g. within 1 hour, 5 hours, 10 hours, 15 hours, 20 hours, and 24 hours) prior to a subject undergoing motor skill restoration therapy. Suitably, the psychedelic compound may be administered within 6 hours (e.g., within 1 hour, 2 hours, 3 hours, 4 hours, 5 hours, and 6 hours) prior to a subject undergoing motor skill restoration therapy. Suitably, empathogenic compound may be administered within 1 hour (e.g., 10 minutes, 20 minutes, 30 minutes, 40 minutes, 50 minutes, and 60 minutes) prior to a subject undergoing motor skill restoration therapy. Suitably, the motor skill restoration therapy may be administered for an additional 2 to 4 weeks after the psychedelic compound is no longer being administered to the subject. Suitably, the motor skill restoration therapy may comprise a rehabilitation program and/or a therapeutic compound. Suitably, the motor skill restoration therapy may include a rehabilitation program such as physical therapy, occupational therapy, or speech therapy.

Suitably, the rehabilitation program may be a robot assisted therapy. Suitably, the robot assisted therapy may be an end-effector type robotic device therapy. Suitably, the robot assisted therapy may be an exoskeleton-type robotic device therapy. Suitably, the rehabilitation program may utilize an approach known as the MindPod Dolphin. Suitably, alternative technologies to be the MindPod Dolphin may be used. Suitably, the rehabilitation program may comprise a 3D virtual reality video game “Bandit's Shark Showdown. Suitably, the rehabilitation program may comprise deep brain stimulation.

Suitably, the rehabilitation program comprises interventions with technologies designed to enhance the intensity of the post-injury rehabilitation.

Suitably, the central nervous system injury may be the result of a stroke (ischemic or hemorrhagic), brain injury or spinal cord injury (e.g. a spinal cord injury caused by a tumor or multiple sclerosis).

Suitably, the empathogenic compound may be for use in combination with an alpha adrenergic drug (e.g., clonidine).

Suitably, the central nervous system injury may impact motor function, such as fine or gross. motor function. Suitably, the motor function may be a fine motor function. Suitably, the fine motor function may be speech.

Suitably, the empathogenic compound may be formulated for administration every 3 to 4 days. Suitably, the psychedelic compound may be formulated for administration every 1 to 2 weeks. Suitably, the empathogenic compound may be formulated for administration every 3 to 4 weeks.

Suitably, the empathogenic compound may be 3,4-methylenedioxymethamphetamine (MDMA) or an analog thereof. Suitably, the empathogenic compound may be a unit dosage form including from 50 to 150 mg (e.g., 55 mg, 60 mg, 70 mg, 80 mg, 90 mg, 100 mg, 110 mg, 120 mg, 130 mg, 140 mg, and 150 mg) of MDMA or a pharmaceutically acceptable salt thereof. Suitably, the unit dosage may be formulated as an oral dose. Suitably, the empathogenic compound may be a unit dosage form including from 50 to 75 mg (e.g., 50±5 mg, 55±5 mg, 60±5 mg, 65±5 mg, 70±5 mg, and 75±5 mg) of MDMA or a pharmaceutically acceptable salt thereof. Suitably, the empathogenic compound may be a unit dosage form including from 75 to 150 mg (e.g., 75±5 mg, 80±5 mg, 85±5 mg, 90±5 mg, 95±5 mg, 100±5 mg, 105±5 mg, 110±5 mg, 115±5 mg, 120±5 mg, 125±5 mg, 130±5 mg, 135±5 mg, 140±5 mg, 145±5 mg, and 150±5 mg) of MDMA or a pharmaceutically acceptable salt thereof. Suitably, the empathogenic compound may be a unit dosage form including 75 mg of MDMA or a pharmaceutically acceptable salt thereof.

Suitably, the empathogenic compound or pharmaceutically acceptable salt thereof may be administered orally, intravenously, by inhalation, by nebulization, by aerosolization, intranasally, intratracheally, intrabronchially, subcutaneously, or intramuscularly.

Suitably, the empathogenic compound may be comprised in a pharmaceutical composition. Accordingly, another aspect provides a pharmaceutical composition comprising an empathogenic compound disclosed herein for use in the treatment of a central nervous system injury or focal diaschisis in a subject.

In another aspect, there is provided dissociative compound for use in the treatment of a central nervous system injury, focal diaschisis or an injury associated with impaired motor function in a subject. Suitably, the focal diaschisis may be caused by a central nervous system injury in a subject. Suitably, the central nervous system injury, focal diaschisis or an injury associated with impaired motor function may be acute, subacute, or chronic. Suitably, the central nervous system injury may a traumatic brain injury or a spinal cord injury (e.g. a spinal cord injury caused by a tumor or multiple sclerosis) or may be caused by a stroke (e.g. ischemic or hemorrhagic). Suitably, the subject may suffer from crossed cerebellar diaschisis (CCD). Suitably, the subject may suffer from stroke, concussion, or chronic traumatic encephalopathy (CTE). Suitably, the subject may be a therapy-refractory subject.

Suitably, the dissociative compound may be administered within 24 hours (e.g. within 1 hour, 5 hours, 10 hours, 15 hours, 20 hours, and 24 hours) prior to a subject undergoing motor skill restoration therapy. Suitably, the psychedelic compound may be administered within 6 hours (e.g., within 1 hour, 2 hours, 3 hours, 4 hours, 5 hours, and 6 hours) prior to a subject undergoing motor skill restoration therapy. Suitably, dissociative compound may be administered within 1 hour (e.g., 10 minutes, 20 minutes, 30 minutes, 40 minutes, 50 minutes, and 60 minutes) prior to a subject undergoing motor skill restoration therapy. Suitably, the motor skill restoration therapy may be administered for an additional 2 to 4 weeks after the dissociative compound is no longer being administered to the subject. Suitably, the motor skill restoration therapy may comprise a rehabilitation program and/or a therapeutic compound. Suitably, the motor skill restoration therapy may include a rehabilitation program such as physical therapy, occupational therapy, or speech therapy.

Suitably, the rehabilitation program may be a robot assisted therapy. Suitably, the robot assisted therapy may be an end-effector type robotic device therapy. Suitably, the robot assisted therapy may be an exoskeleton-type robotic device therapy. Suitably, the rehabilitation program may utilize an approach known as the MindPod Dolphin. Suitably, alternative technologies to be the MindPod Dolphin may be used. Suitably, the rehabilitation program may comprise a 3D virtual reality video game “Bandit's Shark Showdown. Suitably, the rehabilitation program may comprise deep brain stimulation.

Suitably, the rehabilitation program comprises interventions with technologies designed to enhance the intensity of the post-injury rehabilitation.

Suitably, the central nervous system injury may be a stroke (e.g. ischemic or hemorrhagic), brain injury or spinal cord injury (e.g. a spinal cord injury caused by a tumor or multiple sclerosis).

Suitably, the dissociative compound may be for use in combination with an alpha adrenergic drug (e.g., clonidine).

Suitably, the central nervous system injury may impact motor function, such as fine or gross. motor function. Suitably, the motor function may be a fine motor function. Suitably, the fine motor function may be speech

Suitably, the dissociative compound may be formulated for administration every 3 to 4 days. Suitably, the psychedelic compound may be formulated for administration every 1 to 2 weeks. Suitably, the dissociative compound may be formulated for administration every 3 to 4 weeks.

Suitably, the dissociative compound may be ketamine. Suitably, the dissociative compound may be a unit dosage form including from 50 to 200 mg (e.g., 50±10 mg, 60±10 mg, 70±10 mg, 80±10 mg, 90±10 mg, 100±10 mg, 110±10 mg, 120±10 mg, 130±10 mg, 140±10 mg, 150±10 mg, 160±10 mg, 170±10 mg, 180±10 mg, 190±10 mg, and 200±10 mg) of ketamine or a pharmaceutically acceptable salt thereof. Suitably, the unit dosage may be formulated as an intravenous dose. Suitably, the dissociative compound may be a unit dosage form including 100±10 mg of ketamine.

Suitably, the dissociative compound or pharmaceutically acceptable salt thereof may be administered orally, intravenously, by inhalation, by nebulization, by aerosolization, intranasally, intratracheally, intrabronchially, subcutaneously, or intramuscularly.

In another aspect, the disclosure provides the use of a psychedelic compound in the manufacture of a medicament for the treatment of a central nervous system injury, focal diaschisis or an injury associated with impaired motor function in a subject. Suitably, the focal diaschisis may be caused by a central nervous system injury in a subject. Suitably, the central nervous system injury, focal diaschisis or an injury associated with impaired motor function may be acute, subacute, or chronic. Suitably, the central nervous system injury may a traumatic brain injury or a spinal cord injury (e.g. a spinal cord injury caused by a tumor or multiple sclerosis) or may be caused by a stroke (e.g. ischemic or hemorrhagic). Suitably, the subject may suffer from crossed cerebellar diaschisis (CCD). Suitably, the subject may suffer from stroke, concussion, or chronic traumatic encephalopathy (CTE). Suitably, the subject may be a therapy-refractory subject.

Suitably, the psychedelic compound may be administered within 24 hours (e.g. within 1 hour, 5 hours, 10 hours, 15 hours, 20 hours, and 24 hours) prior to a subject undergoing motor skill restoration therapy. Suitably, the psychedelic compound may be administered within 6 hours (e.g., within 1 hour, 2 hours, 3 hours, 4 hours, 5 hours, and 6 hours) prior to a subject undergoing motor skill restoration therapy. Suitably, the psychedelic compound may be administered within 1 hour (e.g., 10 minutes, 20 minutes, 30 minutes, 40 minutes, 50 minutes, and 60 minutes) prior to a subject undergoing motor skill restoration therapy. Suitably, the motor skill restoration therapy is administered for an additional 2 to 4 weeks after the psychedelic compound is no longer being administered to the subject. Suitably, the motor skill restoration therapy may comprise a rehabilitation program and/or a therapeutic compound.

Suitably, the motor skill restoration therapy may include a rehabilitation program such as physical therapy, occupational therapy, or speech therapy. Suitably, the rehabilitation program may comprise deep brain stimulation.

Suitably, the central nervous system injury may the result of a stroke, a brain injury, or a spinal cord injury (e.g. a spinal cord injury caused by a tumor or multiple sclerosis).

Suitably, the psychedelic compound may be for use in combination with an alpha adrenergic drug (e.g., clonidine).

Suitably, the central nervous system injury may impact motor function, such as fine or gross motor function. Suitably, the motor function may be a fine motor function. Suitably, the fine motor function may be speech

Suitably, the psychedelic compound may be formulated for administration every 3 to 4 days. Suitably, the psychedelic compound may be formulated for administration every 1 to 2 weeks. Suitably, the psychedelic compound may be formulated for administration every 3 to 4 weeks.

Suitably, the psychedelic compound may be a 5-HT_{2A} receptor agonist. In particular embodiments, the 5-HT_{2A} receptor agonist is lysergic acid diethylamide (LSD). Suitably, the psychedelic compound may be a unit dosage form including from 5 to 250 μg (e.g., 10 μg, 25 μg, 50 μg, 100 μg, 125 μg, 150 μg, 175 μg, 200 μg, 225 μg, and 250 μg) of lysergic acid diethylamide or a pharmaceutically acceptable salt thereof. Suitably, the unit dosage may be an oral dose. Suitably, the unit dosage form of the psychedelic compound may comprise from 50 to 250 μg (e.g., 10 μg, 25 μg, 50 μg, 100 μg, 125 μg, 150 μg, 175 μg, 200 μg, 225 μg, and 250 μg) of LSD or a pharmaceutically acceptable salt thereof. Suitably, the unit dosage form of the psychedelic compound may comprise from 5 to 30 μg (e.g., 5±5 μg, 10±5 μg, 15±5 μg, 20±5 μg, 25±5 μg, and 30±5 μg) of LSD or a pharmaceutically acceptable salt thereof. Suitably, the unit dosage form of the psychedelic compound may comprise 10±5 μg of LSD or a pharmaceutically acceptable salt thereof. Suitably, the 5-HT_{2A} receptor agonist may be psilocybin or psilocin. Suitably, the psychedelic compound may be a unit dosage form including from 1 to 40 mg (e.g., 2±1 mg, 3±1 mg, 4±1 mg, 5±1 mg, 6±1 mg, 7±1 mg, 8±1 mg, 9±1 mg, 10±5 mg, 15±5 mg, 20±5 mg, 25±5 mg, 30±5 mg, 35±5 mg, and 40±5 mg) of psilocybin or psilocin, or a pharmaceutically acceptable salt thereof. Suitably, the psychedelic compound may be a unit dosage form including from 1 to 10 mg (e.g., 2±1 mg, 3±1 mg, 4±1 mg, 5±1 mg, 6±1 mg, 7±1 mg, 8±1 mg, 9±1 mg, and 10±1 mg) of psilocybin or psilocin, or a pharmaceutically acceptable salt thereof. Suitably, the psychedelic compound may be a unit dosage form including from 5 to 40 mg (e.g., 5±5 mg, 10±5 mg, 15±5 mg, 20±5 mg, 25±5 mg, 30±5 mg, 35±5 mg, and 40±5 mg) of psilocybin or psilocin, or a pharmaceutically acceptable salt thereof.

Suitably, the psychedelic compound or pharmaceutically acceptable salt thereof may be administered orally, intravenously, by inhalation, by nebulization, by aerosolization, intranasally, intratracheally, intrabronchially, subcutaneously, or intramuscularly.

Suitably, the psychedelic compound may be comprised in a pharmaceutical composition. Accordingly, another aspect provides use of a pharmaceutical composition comprising a psychedelic compound disclosed herein in the manufacture of a medicament treatment of a central nervous system injury or focal diaschisis in a subject.

In another aspect, there is provided use of an empathogenic compound in the manufacture of a medicament for the treatment of a central nervous system injury, focal diaschisis or an injury associated with impaired motor function in a subject. Suitably, the focal diaschisis may be caused by a central nervous system injury in a subject. Suitably, the central nervous system injury, focal diaschisis or an injury associated with impaired motor function may be acute, subacute, or chronic. Suitably, the central nervous system injury may a traumatic brain injury or a spinal cord injury (e.g. a spinal cord injury caused by a tumor or multiple sclerosis) or may be caused by a stroke (e.g. ischemic or hemorrhagic). Suitably, the subject may suffer from crossed cerebellar diaschisis (CCD). Suitably, the subject may suffer from stroke, concussion, or chronic traumatic encephalopathy (CTE). Suitably, the subject may be a therapy-refractory subject.

Suitably, the empathogenic compound may be administered within 24 hours (e.g. within 1 hour, 5 hours, 10 hours, 15 hours, 20 hours, and 24 hours) prior to a subject undergoing motor skill restoration therapy. Suitably, the empathogenic compound may be administered within 6 hours (e.g., within 1 hour, 2 hours, 3 hours, 4 hours, 5 hours, and 6 hours) prior to a subject undergoing motor skill restoration therapy. Suitably, empathogenic compound may be administered within 1 hour (e.g., 10 minutes, 20 minutes, 30 minutes, 40 minutes, 50 minutes, and 60 minutes) prior to a subject undergoing motor skill restoration therapy. Suitably, the motor skill restoration therapy is administered for an additional 2 to 4 weeks after the empathogenic compound is no longer being administered to the subject. Suitably, the motor skill restoration therapy may comprise a rehabilitation program and/or a therapeutic compound. Suitably, the motor skill restoration therapy may include a rehabilitation program such as physical therapy, occupational therapy, or speech therapy.

Suitably, the central nervous system injury may the result of a stroke, a brain injury, or a spinal cord injury (e.g. a spinal cord injury caused by a tumor or multiple sclerosis).

Suitably, the empathogenic compound may be for use in combination with an alpha adrenergic drug (e.g., clonidine).

Suitably, the central nervous system injury may impact motor function, such as fine or gross motor function. Suitably, the motor function may be a fine motor function. Suitably, the fine motor function may be speech

Suitably, the empathogenic compound may be formulated for administration every 3 to 4 days. Suitably, the empathogenic compound may be formulated for administration every 1 to 2 weeks. Suitably, the empathogenic compound may be formulated for administration every 3 to 4 weeks.

Suitably, the empathogenic compound may be 3,4-methylenedioxymethamphetamine (MDMA) or an analog thereof. Suitably, the empathogenic compound may be a unit dosage form including from 50 to 150 mg (e.g., 55 mg, 60 mg, 70 mg, 80 mg, 90 mg, 100 mg, 110 mg, 120 mg, 130 mg, 140 mg, and 150 mg) of MDMA or a pharmaceutically acceptable salt thereof. Suitably, the unit dosage may be formulated as an oral dose. Suitably, the empathogenic compound may be a unit dosage form including from 50 to 75 mg (e.g., 50±5 mg, 55±5 mg, 60±5 mg, 65±5 mg, 70±5 mg, and 75±5 mg) of MDMA or a pharmaceutically acceptable salt thereof. Suitably, the empathogenic compound may be a unit dosage form including from 75 to 150 mg (e.g., 75±5 mg, 80±5 mg, 85±5 mg, 90±5 mg, 95±5 mg, 100±5 mg, 105±5 mg, 110±5 mg, 115±5 mg, 120±5 mg, 125±5 mg, 130±5 mg, 135±5 mg, 140±5 mg, 145±5 mg, and 150±5 mg) of MDMA or a pharmaceutically acceptable salt thereof. Suitably, the empathogenic compound may be a unit dosage form including 75 mg of MDMA or a pharmaceutically acceptable salt thereof.

Suitably, the empathogenic compound or pharmaceutically acceptable salt thereof may be administered orally, intravenously, by inhalation, by nebulization, by aerosolization, intranasally, intratracheally, intrabronchially, subcutaneously, or intramuscularly.

Suitably, the empathogenic compound may be comprised in a pharmaceutical composition. Accordingly, another aspect provides use of a pharmaceutical composition comprising an empathogenic compound disclosed herein in the manufacture of a medicament for the treatment of a central nervous system injury or focal diaschisis in a subject.

In another aspect, the disclosure provides the use of a dissociative compound in the manufacture of a medicament for the treatment of a central nervous system injury, focal diaschisis or an injury associated with impaired motor function in a subject. Suitably, the focal diaschisis may be caused by a central nervous system injury in a subject. Suitably, the central nervous system injury, focal diaschisis or an injury associated with impaired motor function may be acute, subacute, or chronic. Suitably, the central nervous system injury may a traumatic brain injury or a spinal cord injury (e.g. a spinal cord injury caused by a tumor or multiple sclerosis) or may be caused by a stroke (e.g. ischemic or hemorrhagic). Suitably, the subject may suffer from crossed cerebellar diaschisis (CCD). Suitably, the subject may suffer from stroke, concussion, or chronic traumatic encephalopathy (CTE). Suitably, the subject may be a therapy-refractory subject.

Suitably, the dissociative compound may be administered within 24 hours (e.g. within 1 hour, 5 hours, 10 hours, 15 hours, 20 hours, and 24 hours) prior to a subject undergoing motor skill restoration therapy. Suitably, the dissociative compound may be administered within 6 hours (e.g., within 1 hour, 2 hours, 3 hours, 4 hours, 5 hours, and 6 hours) prior to a subject undergoing motor skill restoration therapy. Suitably, the dissociative compound may be administered within 1 hour (e.g., 10 minutes, 20 minutes, 30 minutes, 40 minutes, 50 minutes, and 60 minutes) prior to a subject undergoing motor skill restoration therapy. Suitably, the motor skill restoration therapy is administered for an additional 2 to 4 weeks after the dissociative compound is no longer being administered to the subject. Suitably, the motor skill restoration therapy may comprise a rehabilitation program and/or a therapeutic compound. Suitably, the motor skill restoration therapy may include a rehabilitation program such as physical therapy, occupational therapy, or speech therapy.

Suitably, the central nervous system injury may be a stroke, brain injury or spinal cord injury (e.g. a spinal cord injury caused by a tumor or multiple sclerosis).

Suitably, the dissociative compound may be for use in combination with an alpha adrenergic drug (e.g., clonidine).

Suitably, the central nervous system injury may impact motor function, such as fine or gross. motor function. Suitably, the motor function may be a fine motor function. Suitably, the fine motor function may be speech

Suitably, the dissociative compound may be formulated for administration every 3 to 4 days. Suitably, the dissociative compound may be formulated for administration every 1 to 2 weeks. Suitably, the empathogenic compound may be formulated for administration every 3 to 4 weeks.

Suitably, the dissociative compound may be ketamine. Suitably, the dissociative compound may be a unit dosage form including from 50 to 200 mg (e.g., 50±10 mg, 60±10 mg, 70±10 mg, 80±10 mg, 90±10 mg, 100±10 mg, 110±10 mg, 120±10 mg, 130±10 mg, 140±10 mg, 150±10 mg, 160±10 mg, 170±10 mg, 180±10 mg, 190±10 mg, and 200±10 mg) of ketamine or a pharmaceutically acceptable salt thereof. Suitably, the unit dosage may be formulated as an intravenous dose. Suitably, the dissociative compound may be a unit dosage form including 100±10 mg of ketamine.

Suitably, the ketamine compound or pharmaceutically acceptable salt thereof may be administered orally, intravenously, by inhalation, by nebulization, by aerosolization, intranasally, intratracheally, intrabronchially, subcutaneously, or intramuscularly.

Suitably, the ketamine compound may be comprised in a pharmaceutical composition. Accordingly, another aspect provides use of a pharmaceutical composition comprising an empathogenic compound disclosed herein in the manufacture of a medicament for the treatment of a central nervous system injury or focal diaschisis in a subject.

### Definitions

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

Terms such as “a”, “an,” and “the” are not intended to refer to only a singular entity but include the general class of which a specific example may be used for illustration.

As used herein, the term “about” refers to a value that is within 10% above or below the value being described.

The term “administration” or “administering” refers to a method of giving a dosage of a pharmaceutical composition to a patient, where the method is systemic, e.g., oral, topical, transdermal, by inhalation, intravenous, intraperitoneal, intracerebroventricular, intrathecal, or intramuscular. The preferred method of administration can vary depending on various factors, e.g., the components of the pharmaceutical composition, site of administration, and severity of the symptoms being treated.

As used herein, an “acute central nervous system injury” refers to a central nervous system injury that occurs suddenly and which the onset symptoms or immediate complications may require urgent medical care and that has occurred within the past 7 days.

As used herein, a “subacute central nervous system injury” refers to a central nervous system injury that persists, at least partially, over a period greater than 7 days from onset and extending to 6 months and requires either ongoing medical attention and/or limits the activities of daily life.

As used herein, a “chronic central nervous system injury” refers to a central nervous system injury that persists, at least partially, greater than a period of 6 months from onset and requires either ongoing medical attention and/or limits the activities of daily life. In some instances, a central nervous system injury (e.g., stroke, TBI, or spinal cord injury) is characterized herein as a chronic central nervous system injury (e.g., chronic stroke, chronic TBI, or chronic spinal cord injury) if the patient experiences an initial recovery of the central nervous system injury followed by a plateau in recovery, wherein the injury is not fully resolved at 6 months.

As used herein the term “central nervous system injury” refers to an injury to the brain or spinal cord. A central nervous system injury includes stroke, cerebrovascular accident (CVA), transient ischemic attack (TIA), traumatic brain injury (TBI), spinal cord injury, and in certain cases of brain tumors and amyotrophic lateral sclerosis (ALS). Central nervous system injury due to stroke occurs due to reduced perfusion to a brain region. Traumatic brain injury (TBI), also known as intracranial injury, occurs when an external force traumatically injures the brain. Spinal cord injury (SCI) occurs when an external force causes damage to any part of the spinal cord or nerves at the end of the spinal canal. Central nervous system injuries can range from mild to severe with symptoms including recurrent headaches, paresthesia, muscle weakness, numbness, memory loss, impaired mental ability, tremors, seizures, impaired speech, and loss of motor functions Suitably, a “central nervous system injury” as defined herein may be an injury that has resulted in impaired motor function.

As used herein, the term “critical window” refers a window of time of heightened brain plasticity following a central nervous system injury, also called critical or “sensitive periods”. A critical window is the time when environmental input (e.g., motor skill restoration therapy) is required for the proper modification of a particular brain circuit such that the motor skill can be relearned after a loss of the motor skill resulting from the central nervous system injury. If the circuit is left unstimulated, the brain function served by that circuit will be permanently compromised, preventing the subject from regaining lost skills (e.g., fine and gross motor skills) and becoming refractory to therapy. For a subject that has experienced trauma or stroke, the most efficient critical window for functional recovery is typically expected between the time of injury to 6 months.

As used herein, the terms “dosage” and “unit dose” when used in reference to a therapeutic composition refer to physically discrete units suitable as unitary dosage for the subject, each unit containing a predetermined quantity of active material calculated to produce the desired therapeutic effect in association with the required diluent, i.e., carrier, or vehicle.

As used herein, a “5-HT_{2A} agonist” refers to a compound that increases the activity of a 5-hydroxytryptamine 2A receptor. Examples of such agonists include psilocybin or psilocin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine; ayahuasca; 3,4,5-trimethoxyphenethylamine (mescaline); 5-methoxy-N,N-dimethyltryptamine (5-meo-DMT); ibogaine; a compound of formula (I), wherein R^{α}, R^{N}₁, R^{N}₂, R⁴, and/or R⁵ are selected from the group consisting of C, CH₃, OH, F, OCH₃, and H; a compound of formula (II), wherein R^{α}, R^{β}, R², R³, R⁴, R⁵, R⁶, and/or R^{N} are selected from the group consisting of OCH₃, CH₃, SCH₃, Br, I, CH₂CH(CH₃)₂, and H; or a compound of formula (III), wherein R¹, R², and/or R³ are selected from the group consisting of CH₂CH₃, CH(CH₃)CH₂CH₃, CH(CH₃)CH₂CH₂CH₃, C₂H₅, CH₂CH₂CH₃, CH(CH₃)₂, and H. Compounds of formulas (I)-(III) can be synthesized using methods known in the art, including, for example, procedures described in Kornfeld et al. (J. Am. Chem. Soc. 1954, 76(20):5256-5257), Kornfeld et al. (J. Am. Chem. Soc. 1956, 78(13):3087-3114), Marino et al. (J. Org. Chem. 1995, 60(9):2704-2713), and Tsao (J. Am. Chem. Soc. 1951, 73(11):5495-5496).

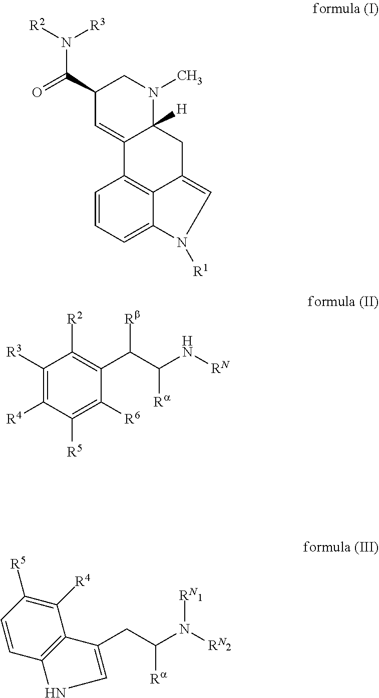

As used herein, the term “month” refers to 30 days±2 days.

As used herein, the term “motor function” refers to both fine and gross motor functions. Gross motor skills refer to movements of the large muscles of the arms, legs, and/or torso. Gross motor functions include but are not limited to, for example, running, jumping, sliding, swimming, throwing, catching, kicking. Fine motor skills refer to the coordination of small muscles, usually in the hands and/or wrists. Fine motor functions include but are not limited to, for example, drawing, writing, speaking, squeezing, fastening buttons or snaps, zipping a zipper, cutting, twisting, brushing hair, knitting, playing a musical instrument, and bathing. Motor function may be assessed by clinician using a physical exam, using the Bruininks Motor Ability Test, the McCarron Assessment of Neuromuscular Development, the Tufts Assessment of Motor Performance, the Zurich Neuromotor Assessment, a kinematic movement assessment, the Fugl-Meyer Assessment of Motor Recovery, the Action Research Arm Test (ARAT), the Chedoke Arm and Hand Activity Inventory (CAHAI), the Tegner Activity Scale (TAS), the Stroke Rehabilitation Assessment of Movement Measurement (STREAM), the Tardieu Scale, Motor Assessment Scale, the Scale of Contraversive Pushing, the Elderly Mobility Scale, Neuromuscular Recovery Scale, the Postural Assessment Scale for Stroke, the Burke Lateropulsion Scale, the Stroke Impact Scale, the Visual Vertigo Analogue Scale, the Berg Balance Scale, the Trunk Impairment Scale, the Pittsburgh Rehabilitation Participation Scale, the Clinical Outcome Variables Scale, the Community Balance and Mobility Scale, the Scale for Assessment and Rating of Ataxia, the Guy's Neurological Disability Scale, the Modified Gait Efficiency Scale, the Lower Extremity Functional Scale, or the Functional Mobility Scale. Suitably, in some aspects, motor function may be assessed using a kinematic movement assessment.

As used herein, the term “motor skill restoration therapy” refers to therapy used to improve muscle function and restore a motor skill that was lost as a result of an injury (e.g., stroke, traumatic brain injury, and spinal cord injury). Motor skill restoration therapy includes physical therapy, occupational therapy, speech therapy, and strength training. The motor still restoration therapy may be administered by a clinician, or the motor skill restoration therapy may be a robot assisted therapy, which may be an end-effector type robot device therapy or an exoskeleton type robot device therapy. The robot assisted may include video games and/or virtual reality environments.

As used herein, the term “pharmaceutically acceptable salt” refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid or inorganic acid.

As used herein, the terms “pharmacologically effective amount,” “therapeutically effective amount,” and the like, when used in reference to a therapeutic composition, refer to a quantity sufficient to, when administered to the subject, including a mammal, for example a human, effect beneficial or desired results, such as clinical results. For example, in the context of treating a central nervous system injury, described herein, these terms refer to an amount of the composition sufficient to achieve a treatment response as compared to the response obtained without administration of the composition. The quantity of a given composition described herein that will correspond to such an amount may vary depending upon various factors, such as the given agent, the pharmaceutical formulation, the route of administration, the type of disease or disorder, the identity of the subject (e.g., age, sex, weight) or host being treated, and the like. An “effective amount,” “pharmacologically effective amount,” or the like, of a composition of the present disclosure, also include an amount that results in a beneficial or desired result in a subject as compared to a control (e.g., restoration of a motor function).

As used herein, a “psychedelic agent” refers to a compound capable of inducing an altered state of consciousness, i.e., a marked deviation in the subjective experience or psychological functioning of a normal individual from his or her usual waking consciousness. Altered states of consciousness can be monitored, evaluated, and/or quantified using any of a variety of methods known in the art including, without limitation, Dittrich's APZ (Abnormal Mental States) questionnaire, and its revised versions, OAV and 5D-ASC (see, for example, Dittrich et al., A Pharmacopsychiatry 1998, 31:80; Studerus et al., PLoS ONE 2010, 5). Psychedelic agents include 5-HT_{2A} agonists (e.g., lysergic acid diethylamide (LSD), empathogenic agents (i.e., serotonin (5-HT) releasing agents; e.g., MDMA), and dissociative agents (i.e., N-Methyl-D-aspartate (NMDA) receptor agonists; e.g., ketamine).

As used herein, the term “subacute central nervous system injury” refers to an injury that to the central nervous system that has that occurred more than 7 days prior and less than 6 months prior.

As used herein, the terms “treat,” “treating,” or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., restoring motor function).

As used herein, the terms “therapy-refractory subject” or “recovery-refractory subject” are interchangeable and refer to a subject with a central nervous system injury, wherein the subject experiences symptoms (e.g., reduced motor skills) or otherwise presents indicators (e.g., as assessed by a clinician) of the central nervous system injury after administration of a standard motor skill restoration therapy in the absence of psychedelic, empathogenic, or dissociative compound treatment. In some embodiments, a therapy-refractory subject does not show an improvement in motor function as a result of being administered a standard motor skill restoration therapy following an injury to the central nervous system. In other embodiments, a therapy-refractive subject shows an incomplete improvement in motor function as a result of being administered a standard motor skill restoration therapy following an injury to the central nervous system (e.g., the subject's response to the standard motor skill restoration therapy has plateaued, e.g., as assessed by a clinician). A subject can become a therapy-refractory subject as a result of closure of the critical window for brain plasticity. For example, the most well characterized example is for post-stroke patients who fail to show meaningful motor recovery 6 months post injury (Joregenson et al 1999). Although there may be some motor recovery in a subset of patients 6 months post-stroke, the recovery is minimal. Further, most upper extremity motor recovery can be predicted using biomarkers. As such, post-stroke motor recovery follows a natural time-course and can be further predicted by well-characterized biomarkers.

Other features and advantages of the invention will be apparent from the following Detailed Description, Examples, and Claims.

### DETAILED DESCRIPTION

The invention provides psychedelic compounds and empathogenic compounds for use in the treatment of a central nervous system injury, focal diaschisis, or an injury associated with impaired motor function in a subject, and methods for improving motor function in a therapy refractory subject with a central nervous system injury (e.g., a chronic central nervous system injury or a subacute central nervous system injury), including a stroke, a traumatic brain injury, and a spinal cord injury, by administering a psychedelic, empathogenic, or dissociative compound to the subject followed by administering a motor skill restoration therapy. The compounds and pharmaceutical compositions for use in accordance with the invention and methods of the invention result in a reopening of the critical window for learning motor functions, which therefore allows for motor function to be improved in subjects that were previously refractory to therapy. This invention also provides new methods for improving motor function in a subject with an central nervous system injury (e.g., an acute central nervous system injury and an subacute central nervous system injury) by administering a psychedelic, empathogenic, or dissociative compound to the subject within 3 months of the injury occurring, followed by administering a motor skill restoration therapy. The method results in the critical window for relearning motor functions to be extended past the typical amount of time the critical window remains open after sustaining an injury to the central nervous system such that motor function can continue to be improved over a longer period of time. Lastly, the invention provides new methods for treating focal diaschisis caused by a central nervous system injury by administering a psychedelic, empathogenic, or dissociative compound to the subject followed by administering a motor still restoration therapy resulting in improved motor function in the subject.

### Central Nervous System Injuries

Central nervous system injuries include injuries to the brain and/or spinal cord. A central nervous system injury may be sustained from an external force, as in the case of traumatic brain injury and spinal cord injury, or from a physical event internal to the body, such as a stroke. The invention provides psychedelic, empathogenic, or dissociative compounds and methods for improving motor function in a subject who has suffered a central nervous system injury (e.g., an acute central nervous system injury, a subacute central nervous system injury, or a chronic central nervous system injury). The subject may be determined to have a chronic central nervous system injury, including central nervous injuries which persist for a time period of 6 months or longer. The subject may also be determined to have an acute central nervous system injury, which has occurred within the past 7 days. The subject may also be determined to have a subacute central nervous system injury, which has occurred within between 7 days and 6 months prior. The invention provides psychedelic, empathogenic, or dissociative compounds and methods for improving motor function in these subjects by either reopening the critical window for relearning motor function or extending the critical window for relearning motor functions. After a subject sustains an injury to the central nervous system, a critical window for relearning motor skills begins where the neuroplasticity of the brain is increased, thereby allowing the subject to relearn motor skills that were impaired as a result of the central nervous system injury. However, critical window lasts only a fixed amount of time before the window closes. Once closed, the neuroplasticity of the brain decreases resulting in little to no improvement in motor function despite continued administration of motor skill restoration therapy. The psychedelic, empathogenic, or dissociative compounds and methods described herein may reopen the critical window for relearning motor functions or may extend the amount of time the critical window is open.

The central nervous injury sustained by the subject may be a cerebrovascular accident, such as a stroke which occurs when the blood supply to a part of the brain is interrupted (i.e., ischemic stroke) by obstruction of a blood vessel by a blood clot, an embolism, systemic hypoperfusion, or cerebral venous sinus thrombosis or when a blood vessel in the brain bursts and releases blood into the spaces surrounding the brain cells (i.e., hemorrhagic stroke) as a result of an intracerebral or a subarachnoid hemorrhage. Depending on the area of the brain affected by the stroke, the symptoms of a stroke may include numbness or weakness, especially on one side of the body corresponding to the contralateral side of the stroke, muscle flaccidity or spasticity, confusion, trouble understanding or producing speech, impaired vision in both eyes, impaired mobility, dizziness, severe headache, or loss of balance or coordination. Stroke may be diagnosed using several techniques, such as, e.g., neurological examination, blood testing, computed tomography (CT) scan, magnetic resonance imaging (MRI) scan, Doppler ultrasound, and arteriography. A stroke can cause damage to the central nervous system resulting in a loss of both fine and gross motor functions. In some instances of any of the embodiments of the invention, the acute central nervous system injury results from a stroke. In some instances of any of the embodiments of the invention, the subacute central nervous system injury results from a stroke. In other instances, the chronic central nervous system injury results from a stroke. In some instances, the central nervous system injury is a cerebrovascular accident (CVA). In other instances, the central nervous system injury is a transient ischemic attack (TIA).

The central nervous system injury sustained by the subject may also be a spinal cord injury which occurs as a result of an external trauma to the spinal cord. A spinal cord injury may result from an injury to the spine itself or the nerves at the end of the spinal canal. As a result of the spinal cord injury, the subject may experience a loss of function, including loss of mobility and/or feeling. A spinal cord injury may be diagnosed by a clinician using a neurological exam, CT scan, a magnetic resonance imaging (MRI) scan, a myelogram, a somatosensory evoked potential test, or an x-ray of the spine. In some instances of any of the embodiments of the invention, the acute central nervous system injury is a spinal cord injury. In some instances of any of the embodiments of the invention, the subacute central nervous system injury is a spinal cord injury. In other instances, the chronic central nervous system injury is a spinal cord injury.

Additionally, the central nervous system injury sustained by the subject may be a traumatic brain injury (TBI), which is a disruption in the normal function of the brain. A TBI occurs as a result of an external force to the brain. The severity of a TBI may range from mild to severe with symptoms such as headache, blurry vision, nausea, dizziness, balance problems, slurred speech, impaired motor function, seizures, or loss of consciousness. A traumatic brain injury may be diagnosed by a clinician using a neurological exam, a CT scan, or an MRI scan. In some instances of any of the embodiments of the invention, the acute central nervous system injury is a TBI. In some instances of any of the embodiments of the invention, the subacute central nervous system injury is a TBI. In other instances, the chronic central nervous system injury is a TBI.

In certain instances, the central nervous system injury may be caused by a brain tumor or amyotrophic lateral sclerosis (ALS).

As a result of a central nervous system injury, the subject may develop focal diaschisis. The subject with focal diaschisis may suffer from, crossed cerebellar diaschisis. The subject with focal diaschisis may also suffer from a stroke, a concussion or chronic traumatic encephalopathy (CTE). Focal diaschisis results in a change in function of brain due to injury to the brain. The disruption in brain function may occur in an area of the brain that is remote from the focal lesion caused by the injury to the brain. The healthy area of the brain begins to show a loss of function as a result of the neural connectivity to the focal lesion, which may result in symptoms including loss of mobility, impaired motor function, impaired speech, headaches, dizziness, and impaired cognitive function. Focal diaschisis is diagnosed by a clinician using a neurological exam or an MRI scan.

### Motor Function

The invention features psychedelic, empathogenic, or dissociative compounds and methods for improving motor function in a subject diagnosed with a central nervous system injury by administering a psychedelic agent, including a 5-HT_{2A} receptor agonist, an empathogenic compound, or a dissociative compound, followed by administration of a motor skill restoration therapy, resulting in improved motor function. Central nervous system injury (e.g., stroke, traumatic brain injury (TBI), and spinal cord injury (SCI)) may result in loss or impairment of motor functions in subject. The motor function affected by the central nervous system injury may be a gross motor function, including walking, running, jumping, or throwing. Additionally or alternatively, the motor function affected by the central nervous system injury may be a fine motor function, including writing, speaking, and squeezing. In particular embodiments, speech is the motor function that is improved by the administration of a psychedelic, an empathogenic, or a dissociative compound before a subject is administered a motor function rehabilitation therapy.

Motor functions may be assessed using by clinician using a physical exam, a neurological exam, and/or using an assessment test including the Bruininks Motor Ability Test, the McCarron Assessment of Neuromuscular Development, the Tufts Assessment of Motor Performance, or the Zurich Neuromotor Assessment, a Fugl-Meyer Assessment, an Action Research Arm Test, the Tegner Activity Scale (TAS), the Chedoke Arm and Hand Activity Inventory, the Stroke Rehabilitation Assessment of Movement Measurement (STREAM), the Tardieu Scale, or a kinematic assessment. Suitably, motor functions may be assessed by kinematic assessment,

Motor functions may be improved or restored after a central nervous system injury by administering a motor skill restoration therapy including a rehabilitation program or a therapeutic compound. A motor skill restoration therapy may be a rehabilitation program, including physical therapy, occupational therapy, and/or speech therapy.

### Psychedelic Compounds

The invention features methods for improving motor function in a subject by administering a psychedelic, empathogenic, or dissociative agent. The invention further provides a psychedelic, empathogenic, or dissociative compound for use in the treatment of a central nervous system injury, focal diaschisis or an injury associated with impaired motor function in a subject.

Psychedelic agents useful as part of the invention include any compound capable of inducing an altered state of consciousness, i.e., a marked deviation in the subjective experience or psychological functioning of a normal individual from his or her usual waking consciousness. Psychedelic agents include 5-HT_{2A} agonists (e.g., lysergic acid diethylamide (LSD)), empathogenic agents (i.e., serotonin (5-HTreleasers, N-Methyl-D-aspartate (NMDA) receptor agonists) releasing agents; e.g., 3,4-methylenedioxymethamphetamine (MDMA)), and dissociative agents (e.g., ketamine), and oneirophrenic agents (e.g. ibogaine, noribogaine, salvinorin A). The psychedelic compound administered to the subject may be a 5-HT_{2A} receptor agonist. In particular embodiments, the 5-HT_{2A} receptor agonist is LSD. In other embodiments, the 5-HT_{2A} receptor agonist is psilocybin or psilocin. The empathogenic compound administered to the subject may be MDMA. The dissociative compound administered to the subject may be ketamine.

5-HT_{2A} agonists include psilocybin, psilocin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2′S,4′S)-(+)-9,10-Didehydro-6-methylergoline-8β-(trans-2,4-dimethylazetidide); 2C-BCB (4-Bromo-3,6-dimethoxybenzocyclobuten-1-yl) methylamine; ayahuasca; 3,4,5-trimethoxyphenethylamine (mescaline); and 5-methoxy-N,N-dimethyltryptamine (5-meo-DMT).

In some embodiments, the 5-HT_{2A} agonist of the invention includes a compound having the following chemical formula (I):

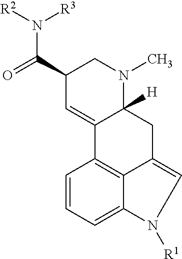

where exemplary values of the R groups in the above substituted chemical structure can be one or more of those represented in the following table:

| Name | R¹ | R² | R³ |
|---|---|---|---|
| LSD | H | CH₂CH₃ | CH₂CH₃ |
| Ergine | H | H | H |
| R-2-butyl | H | H | CH(CH₃)CH₂CH₃ |
| R-2-pentylamine | H | H | CH(CH₃)CH₂CH₂CH₃ |
| Analog of ergine | H | C₂H₅ | H |
| Analog of ergine | H | H | C₂H₅ |
| LSD | H | C₂H₅ | C₂H₅ |
| Analog of ergine | H | C₂H₅ | CH₂CH₂CH₃ |
| Analog of ergine | H | C₂H₅ | CH(CH₃)₂ |
| Analog of ergine | H | CH₂CH₂CH₃ | H |
| Analog of ergine | H | H | CH₂CH₂CH₃ |
| Analog of ergine | H | CH₂CH₂CH₃ | CH₂CH₂CH₃ |
| Analog of ergine | H | CH₂CH₂CH₃ | C₂H₅ |
| Analog of ergine | H | CH₂CH₂CH₃ | CH(CH₃)₂ |
| Analog of ergine | H | CH(CH₃)₂ | H |
| Analog of ergine | H | H | CH(CH₃)₂ |
| Analog of ergine | H | CH(CH₃)₂ | CH(CH₃)₂ |
| Analog of ergine | H | CH(CH₃)₂ | C₂H₅ |
| Analog of ergine | H | CH(CH₃)₂ | CH₂CH₂CH₃ |

In some embodiments, R¹ of formula (I) can be H, C₁-C₆-alkyl, OH, O—(C₁-C₆-alkyl), halogen, or C₁-C₄-haloalkyl; R² of formula (I) can be H, C₁-C₆-alkyl, OH, O—(C₁-C₆-alkyl), halogen, or C₁-C₄-haloalkyl; and R³ of formula (I) can be H, C₁-C₆-alkyl, OH, O—(C₁-C₆-alkyl), halogen, or C₁-C₄-haloalkyl.

In some embodiments, the 5-HT_{2A} agonist of the invention includes a compound having the following chemical formula (II):

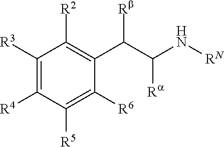

where exemplary values of the R groups in the above substituted chemical structure can be one or more of those represented in the following table:

| Name | R² | R³ | R⁴ | R⁵ | R^{α} | R^{β} |
|---|---|---|---|---|---|---|
| Mescaline | | OCH₃ | OCH₃ | OCH₃ | | |
| TMA | | OCH₃ | OCH₃ | OCH₃ | CH₃ | |
| TMA-2 | OCH₃ | | OCH₃ | OCH₃ | CH₃ | |
| methoxyDOB | OCH₃ | | Br | OCH₃ | CH₃ | OCH₃ |
| DOM | OCH₃ | | CH₃ | OCH₃ | | |
| DOB | OCH₃ | | Br | OCH₃ | | |
| DOI | OCH₃ | | I | OCH₃ | | |
| Sulfur analog of | | OCH₃ | OCH₃ | SCH₃ | | |
| mescaline | | | | | | |
| Sulfur analog of | | OCH₃ | SCH₃ | OCH₃ | | |
| mescaline | | | | | | |
| DOIB | OCH₃ | | CH₂CH(CH₃)₂ | OCH₃ | CH₃ | |
| DOTFM | OCH₃ | | CF₃ | OCH₃ | CH₃ | |

In some embodiments, R² of formula (II) can be OH, O—(C₁-C₆-alkyl), —O—(C₂-C₆-alkyl)-N(R⁵)₂, or —O—(C₂-C₆-alkyl)-N(R^{x})₃⁺halogen⁻; R³ of formula (II) can be OH, O—(C₁-C₆-alkyl), —O—(C₂-C₆-alkyl)-N(R^{x})₂, or —O—(C₂-C₆-alkyl)-N(R^{x})₃⁺halogen⁻; R⁴ of formula (II) can be halogen, C₁-C₂-haloalkyl, H, C₁-C₆-alkyl, C₁-C₆-alkyl sulfide, OH, O—(C₁-C₆-alkyl), —O—(C₂-C₆-alkyl)-N(R^{x})₂, or —O—(C₂-C₆-alkyl)-N(R^{x})₃⁺halogen⁻; R⁵ of formula (II) can be halogen, C₁-C₂-haloalkyl, H, C₁-C₆-alkyl, C₁-C₆-alkyl sulfide, OH, O—(C₁-C₆-alkyl), —O—(C₂-C₆-alkyl)-N(R^{x})₂, or —O—(C₂-C₆-alkyl)-N(R^{x})₃⁺halogen⁻; R⁶ of formula (II) can be halogen, C₁-C₂-haloalkyl, H, C₁-C₆-alkyl, —S—(C₁-C₆-alkyl), OH, O—(C₁-C₆-alkyl), —O—(C₂-C₆-alkyl)-N(R⁵)₂, or —O—(C₂-C₆-alkyl)-N(R⁵)₃⁺halogen⁻; R^{α} is H, halogen, or C₁-C₆-alkyl; RR of formula (II) can be OH, O—(C₁-C₆-alkyl), —O—(C₂-C₆-alkyl)-N(R⁵)₂, or —O—(C₂-C₆-alkyl)-N(R^{x})₃⁺halogen⁻; R^{N} of formula (II) can be halogen, C₁-C₂-haloalkyl, H, C₁-C₆-alkyl, C₁-C₆-alkyl sulfide, OH, O—(C₁-C₆-alkyl), —O—(C₂-C₆-alkyl)-N(R^{x})₂, or —O—(C₂-C₆-alkyl)-N(R^{x})₃⁺halogen⁻; and R^{x} is independently H or C₁-C₄-alkyl.

In some embodiments, the 5-HT_{2A} agonist of the invention includes a compound having the following chemical formula (III):

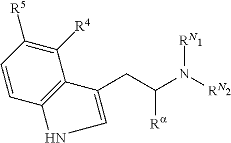

where exemplary values of the R groups in the above substituted chemical structure can be one or more of those represented in the following table:

| Name | R^{N}₁ | R^{N}₂ | R^{α} | R⁴ | R⁵ | R⁶ | R⁷ |
|---|---|---|---|---|---|---|---|
| 6-fluoro-psilocin | C | C | H | OF | H | F | H |
| 7-fluoro-psilocin | C | C | H | OH | H | H | F |
| 4-fluoro-5-methoxy-DMT | C | C | H | F | OCH₃ | H | H |
| 6-fluoro-5-methoxy-DMT | C | C | H | H | OCH₃ | F | H |
| α-Methyl-tryptamine | H | H | CH₃ | H | H | H | H |
| Serotonin | H | H | H | H | OH | H | H |

In some embodiments, R^{N}₁ of formula (III) can be H, C₁-C₆-alkyl, OH, O—(C₁-C₆-alkyl), halogen, or C₁-C₄-haloalkyl; R^{N}₂ of formula (III) can be H, C₁-C₆-alkyl, OH, O—(C₁-C₆-alkyl), halogen, or C₁-C₄-haloalkyl; R^{α} of formula (III) can be H, C₁-C₆-alkyl, OH, O—(C₁-C₆-alkyl), halogen, or C₁-C₄-haloalkyl; R⁴ of formula (III) can be H, C₁-C₆-alkyl, OH, O—(C₁-C₆-alkyl), halogen, or C₁-C₄-haloalkyl; R⁵ of formula (III) can be H, C₁-C₆-alkyl, OH, O—(C₁-C₆-alkyl), halogen, or C₁-C₄-haloalkyl; R⁶ of formula (III) can be H, C₁-C₆-alkyl, OH, O—(C₁-C₆-alkyl), halogen, or C₁-C₄-haloalkyl; and R⁷ of formula (III) can be H, C₁-C₆-alkyl, OH, O—(C₁-C₆-alkyl), halogen, or C₁-C₄-haloalkyl.

### Therapies

The psychedelic, empathogenic, or dissociative compound for use in accordance with the invention and the methods of the invention can be used to improve motor function in subjects diagnosed with a central nervous system injury (e.g., resulting from a stroke, traumatic brain injury, and spinal injury) using a psychedelic (e.g., lysergic acid diethylamide (LSD) and psilocybin or psilocin), empathogenic (e.g., 3,4-methylenedioxymethamphetamine (MDMA)), or dissociative (e.g., ketamine) compound to the subject followed by a motor skill restoration therapy. For example, a psychedelic therapy can be administered to a subject with impaired motor function caused by a central nervous system injury prior to the subject undergoing a motor function restoration therapy, such as physical therapy, occupational therapy, and speech therapy. Suitably, the motor function restoration therapy may comprise deep brain stimulation.

### Patient Population

The psychedelic, empathogenic, or dissociative compound for use in accordance with the invention and the methods of the present invention relate to improving motor function in therapy refractive subjects with a chronic central nervous system injury (e.g., a subacute central nervous system injury or a chronic central nervous system injury) (e.g., stroke, traumatic brain injury, and spinal injury) in order to improve motor function. The subject may be determined to be therapy refractive by a clinician. A candidate for the methods described herein may be a subject who has a central nervous system injury which has persisted for over 3 months and has little to no improvement in motor function as a result of motor function restoration therapy. The subject may be therapy refractive because the critical window for relearning motor functions after a central nervous system injury is closed and therefore the subject's brain now lacks the neuroplasticity required for relearning.

Additionally, the psychedelic, empathogenic, or dissociative compound for use in accordance with the invention and the methods of the present invention relate to improving motor function in a subject with an central nervous system injury (e.g., an acute central nervous system injury and a subacute central nervous system injury) (e.g., stroke, traumatic brain injury, and spinal injury). A candidate for the psychedelic, empathogenic, or dissociative compound for use in accordance with the invention and the methods of the invention may be a subject who has sustained a central nervous system injury within the previous 3 months. As a result of the central nervous system injury, the critical window for relearning motor functions is open and neuroplasticity is increased. The methods described herein may extend the time period of the critical window such that motor function can be improved in the subject for an extended amount of time in comparison to a subject who does not receive a psychedelic, empathogenic, or dissociate compound.

The psychedelic, empathogenic, or dissociative compound for use in accordance with the invention and the methods of the present invention also relate to treating a subject with focal diaschisis resulting from a central nervous system injury to improve motor function. In some embodiments, the subject has suffered a stroke. In certain embodiments, the subject suffered a concussion. In particular embodiments, the subject suffers from chronic traumatic encephalopathy. In some embodiments, the subject suffers from crossed cerebellar diaschisis.

### Psychedelic, Empathogenic, and Dissociative Therapies

Using psychedelic, empathogenic, or dissociative compounds for use in accordance with the invention and the methods of the invention, a psychedelic, empathogenic, or dissociative compound may be administered to the subject prior to administering a motor skill restoration therapy (e.g., physical therapy, occupational therapy, strength training, and speech therapy). The motor skill restoration therapy may be administered to the subject within 6 hours (e.g., within 1 hour, 2 hours, 3 hours, 4 hours, 5 hours, and 6 hours) of the subject being administered the psychedelic compound, empathogenic compound, or dissociative compound. In some embodiments, the motor skill restoration therapy may be administered to the subject within 1 hour (e.g., within 10 minutes, 20 minutes, 30 minutes, 40 minutes, 50 minutes, and 60 minutes) of the subject being administered the psychedelic, empathogenic, or dissociative compound. In certain embodiments, the motor still restoration therapy is continued for an additional 2 to 4 weeks following the final administration of the psychedelic, empathogenic, or dissociative compound. After the initial administration of the psychedelic, empathogenic, or dissociative compound to the subject, the subject may be administered additional doses of the psychedelic, empathogenic, or dissociative compounds prior to, concurrently, or after administration of the motor skill restoration therapy.

The psychedelic, empathogenic, or dissociative compound administered prior to motor function restoration therapy may be administered to the subject every 3 to 4 days. Alternatively, the psychedelic, empathogenic, or dissociative compound may be administered to the subject every 1 to 2 weeks. Alternatively, the psychedelic, empathogenic, or dissociative compound may be administered to the subject every 3 to 4 weeks.

The pharmaceutical composition including the psychedelic, empathogenic, or dissociative compound may be administered as a unit dosage. The unit dosage administered of LSD may be an oral dose. The unit dosage of LSD may also be an intravenous dose. In some embodiments, the subject is administered a unit dosage form containing from 5 to 250 μg (e.g. 10 μg, 20 μg, 50 μg, 100 μg, 150 μg, 200 μg, and 250 μg) of LSD or a pharmaceutically acceptable salt thereof. In certain embodiments, the unit dosage contains from 5 to 30 μg (e.g., 10 μg, 15 μg, 20 μg, 25 μg, and 30 μg) of LSD or a pharmaceutically acceptable salt thereof. In particular embodiments, the pharmaceutical composition is a unit dosage form including 10±5 μg of LSD or a pharmaceutically acceptable salt thereof. The LSD may be administered to the subject orally.

The pharmaceutical composition of psilocybin or psilocin administered to the subject may be a unit dosage form. The unit dosage of psilocybin or psilocin may be an oral dose. The unit dosage of psilocybin or psilocin may also be an intravenous dose. In some embodiments, the subject is administered a unit dosage form containing from 1 mg to 40 mg (e.g. 2 mg, 3 mg, 4 mg, 5 mg, 10 mg, 15 mg, 20 mg, 25 mg, 30 mg, 35 mg, and 40 mg) of psilocybin or psilocin or a pharmaceutically acceptable salt thereof. For example, the unit dosage may contain from 1 mg to 10 mg (e.g., 2 mg, 3 mg, 4 mg, 5 mg, 6 mg, 7 mg, 8 mg, 9 mg, and 10 mg) of psilocybin or psilocin, or a pharmaceutically acceptable salt thereof. In some embodiments, the unit dosage contains from 5 mg to 40 mg (e.g., 10 mg, 15 mg, 20 mg, 25 mg, 30 mg, and 40 mg) of psilocybin or psilocin, or a pharmaceutically acceptable salt thereof. In certain embodiments, the psilocybin or psilocin may be administered to the subject as an intravenous infusion. In some embodiments, the psilocybin or psilocin is administered to the subject orally.

It is contemplated that the psilocybin or psilocin infusion may be administered to the subject at a rate of between 1 and 8 mg of psilocybin or psilocin per hour (e.g., 2±1 mg/hr, 3±1 mg/hr, 4±1 mg/hr, 5±1 mg/hr, 6±1 mg/hr, 7±1 mg/hr, and 8±1 mg/hr) where the infusion lasts between 15 minutes and 4 hours. For example, the psilocybin or psilocin infusion may be administered via a continuous intravenous infusion at a rate of between 4 and 8 mg of psilocybin or psilocin per hour (e.g., 4±1 mg/hr, 5±1 mg/hr, 6±1 mg/hr, 7±1 mg/hr, and 8±1 mg/hr) where the infusion lasts between 15 minutes and 1 hour. In some embodiments, the psilocybin or psilocin infusion may be administered at a rate of 1 mg and 4 mg of psilocybin or psilocin per hour (e.g., 2±1 mg/hr, 3±1 mg/hr, and 4±1 mg/hr) where the infusion lasts between 30 minutes and 4 hours. The rate of administration of the psilocybin or psilocin infusion to the subject may be configured to vary during the period of administration.

The pharmaceutical composition of MDMA administered to the subject may be a unit dosage form. The unit dosage form of MDMA may include from 50 to 150 mg (e.g., 60 mg, 75 mg, 100 mg, 125 mg, and 150 mg) of MDMA or a pharmaceutically acceptable salt thereof. In some embodiments, the unit dosage of MDMA is an oral dose. In certain embodiments, the unit dosage of MDMA is an intravenous dose. The subject may be administered a unit dosage form containing from 50 to 75 mg (e.g., 55 mg, 60 mg, 65 mg, 70 mg, and 75 mg) of MDMA or a pharmaceutically acceptable salt thereof. In particular embodiments, the subject is administered a unit dosage form containing from 75 to 150 mg (e.g., 80 mg, 90 mg, 100 mg, 110 mg, 120 mg, 130 mg, 140 mg, and 150 mg) of MDMA or a pharmaceutically acceptable salt thereof. For example, the subject is administered a unit dosage form of about 75 mg of MDMA. The MDMA may be administered to the subject orally. In some embodiments, the MDMA is deuterated MDMA. In some embodiments, the MDMA is an MDMA analog, for example the MDMA analog may be one or more MDMA analogs selected from the group consisting of: methylone, MDMA Stereoisomer (R-MDMA), 2C-B (4-Bromo-2,5-Dimethoxyphenethylamine), 2C-E (2,5-Dimethoxy-4-ethylphenethylamine), 2C-I (2,5-Dimethoxy-4-iodophenethylamine), 2C-T (2,5-Dimethoxy-4-methylthiophenethylamine). 2C-T-2 (2,5-Dimethoxy-4-ethylthiophenethylamine) and 2C-T-7(2,5-Dimethoxy-4-(n)-propylthiophenethylamine).

The pharmaceutical composition of ketamine administered to the subject may be a unit dosage form. The unit dosage form may include from 50 to 200 mg (e.g., 60 mg, 75 mg, 100 mg, 125 mg, 150 mg, 175 mg, and 200 mg) of ketamine or a pharmaceutically acceptable salt thereof. The unit dosage form may be an oral dose. The unit dosage form may be an intravenous dose. The unit dosage form may also be an intravenous dose. The pharmaceutical composition may be a unit dosage form including 100±10 mg of ketamine or a pharmaceutically acceptable thereof. The ketamine may be administered to the subject as an intravenous infusion.

It is contemplated that the ketamine infusion may be administered to the subject at a rate of between 20 and 150 mg of ketamine per hour (e.g., 20±10 mg/hr, 30±10 mg/hr, 40±10 mg/hr, 50±10 mg/hr, 60±10 mg/hr, 70±10 mg/hr, 80±10 mg/hr, 90±10 mg/hr, 100±10 mg/hr, 110±10 mg/hr, 120±10 mg/hr, 130±10 mg/hr, 140±10 mg/hr, and 150±10 mg/hr). For example, the ketamine infusion may be administered via a continuous intravenous infusion at a rate of between 40 and 100 mg of ketamine per hour (e.g., 40±10 mg/hr, 50±10 mg/hr, 60±10 mg/hr, 70±10 mg/hr, 80±10 mg/hr, 90±10 mg/hr, and 100±10 mg/hr). In some embodiments, the ketamine infusion may be administered at a rate of 40 mg and 60 mg of ketamine per hour (e.g., 40±10 mg/hr, 50±10 mg/hr, and 60±10 mg/hr). In some embodiments, the ketamine infusion may be administered at a rate of between 80 mg and 120 mg of ketamine per hour. In some embodiments, the ketamine infusion is administered at a rate of between 25 mg and 35 mg of ketamine per hour (e.g., 25±1 mg/hr, 26±1 mg/hr, 27±1 mg/hr, 28±1 mg/hr, 29±1 mg/hr, 30±1 mg/hr, 31±1 mg/hr, 32±1 mg/hr, 33±1 mg/hr, 34±1 mg/hr, and 35±1 mg/hr). The rate of administration of the ketamine infusion to the subject may be configured to vary during the period of administration. During the administration of the ketamine infusion, at least 0.5 mg of ketamine may be delivered to the subject per kg of the subject's body mass. The administration of the ketamine infusion may also be performed over a time period of at least an hour.

The psychedelic, empathogenic, or dissociative compound or a pharmaceutically acceptable salt thereof may be administered orally, intravenously, by inhalation, by nebulization, by aerosolization, intranasally, intratracheally, intrabronchially, subcutaneously, or intramuscularly

In any one of the methods described herein the subject may also be administered an alpha adrenergic agonist including clonidine, dexmedetomidine, fadolmidine, guanfacine, xylazine, medetomidine, methyldopa, methylnorepinephrine, norepinephrine, amitraz, detomidine, and lofexidine, in combination with the psychedelic, empathogenic, or dissociative compound. In some embodiments, the alpha adrenergic agonist is clonidine.

In any one of the methods described herein the subject may also be administered a 5-HT3 antagonist including ondansetron, granisetron, dolasetron, palosetron.

### Motor Function Restoration Therapies

Motor functions may be improved or restored after a central nervous system injury by administering a motor skill restoration therapy including a rehabilitation program or a therapeutic compound. A motor skill restoration therapy may be a rehabilitation program, including physical therapy, occupational therapy, and/or speech therapy. The rehabilitation program may be a clinician assisted therapy, or the rehabilitation program may involve the use of the MindPod Dolphin and/or other technology designed to enhance post-neurological injury recovery. The rehabilitation program may comprise deep brain stimulation. The rehabilitation program may be performed for between 10 minutes and 6 hours. In some embodiments, the rehabilitation program may be performed between once a week and 5 times a week.

In some embodiments, the robot assisted therapy may be performed by an end-effector type robot device. In some embodiments, the robot assisted therapy may be performed by a wearable device. For example, the robot assisted therapy may be performed by an exoskeleton type robot device. The robot assisted therapy may aide in gait function. Examples of robot assisted therapies for gait function include but are not limited to a gait trainer or LOKAMAT™, which may be used to provide support in learning to walk, maintaining momentum, and building muscle skills. The robot assisted therapy may assist in upper limb motor function. For example, the robot assisted therapy for upper limb motor function may be MIME, MID-MANUS, Bi-Manu-Track, InMotion, NeReBot, InMotion2, InMotion 2.0 Shoulder/Arm Robot, ARM-Guide, REHA ROB, ARMOR, or T-WREX. The robot assisted therapy may assist in hand motor function. For example, the robot assisted therapy to aide in hand motor function may include the end-effector type devices: pneumatic orthosis, PneuGlove, and Amadeo, or the exoskeleton type devices: HAWARD and Hand Mentor. Hand motor function devices may be used, for example, for training grasp-and-release movement either with real objects or virtual objects in a virtual reality environment. Additionally, the robotic assisted therapy may include an end-effector robot type device in combination with a video game and/or virtual reality environment, for example, the I Am Dolphin video game as described in US 2018/024761, and the JOGO-Gx system, which includes wearable wireless s/EMG sensors and a mobile app that leverages artificial intelligence/machine learning and virtual reality to provide treatment protocols and games that can be adapted for muscle relaxation, movement coordination, and neuro-muscular re-education, all leveraging neural plasticity. In some embodiments, the motor skill restoration therapy may include a biofeedback driven medical device; for example, the biofeedback driven device may include an electromyography-biofeedback driven device.

The motor skill restoration therapy may include transcranial magnetic stimulation. In particular embodiments, the motor skill restoration therapy includes transcranial direct current stimulation.

### Pharmaceutical Compositions

The psychedelic, empathogenic, and dissociative compounds, or pharmaceutically acceptable salts thereof, may be contained in any appropriate amount in any suitable carrier substance and may generally present in an amount of 0.1-95% by weight of the total weight of the composition.

The psychedelic, empathogenic, and dissociative compounds may be formulated for oral use in tablets containing the psychedelic, empathogenic, or dissociative compound in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated, or they may be coated by known techniques, optionally to delay disintegration and absorption in the gastrointestinal tract and thereby providing a sustained action over a longer period. For example, the coating may be adapted to release a psychedelic agent in a predetermined pattern (e.g., in order to achieve a controlled release formulation) or it may be adapted not to release the psychedelic agent until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the psychedelic agent). The coating may be applied on the solid dosage form in a similar manner as that described in Encyclopedia of Pharmaceutical Technology (eds. J. Swarbrick and J. C. Boylan, 1988-1999, Marcel Dekker, New York).

Formulations for oral use may also be presented as chewable tablets, or as hard gelatin capsules wherein the psychedelic agent is mixed with an inert solid diluent (e.g., potato starch, lactose, microcrystalline cellulose, calcium carbonate, calcium phosphate or kaolin), or as soft gelatin capsules wherein the psychedelic compound is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Powders, dispersible powders, or granules suitable for preparation of an aqueous suspension by addition of water are convenient dosage forms for oral administration of psychedelic agents. Formulation as a suspension provides the psychedelic, the empathogenic, or the dissociative compound in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

The pharmaceutical composition may also be administered parenterally by injection, infusion, or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Hayes (Remington: The Science and Practice of Pharmacy, volume I and volume II. Twenty-second edition. Philadelphia, 2012).

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the psychedelic compound, the composition may include suitable parenterally acceptable carriers and/or excipients. The psychedelic, the empathogenic, or the dissociative compound may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions according to the invention may be in the form suitable for sterile injection. To prepare such a composition, the psychedelic, empathogenic, or dissociative compound is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### EXAMPLES

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1: Improving Motor Function in a Therapy Refractory Subject Diagnosed with a Chronic Traumatic Brain Injury

A subject suffering from a chronic traumatic brain injury (TBI) is treated with lysergic acid diethylamide (LSD). The subject is first diagnosed by a clinician with a TBI, resulting from an external force traumatic injury, using a neurological exam and an MRI. As a result of the TBI, the subject has impaired motor functions including an impaired ability to walk. The subject's motor function is assessed using the Bruininks Motor Ability Test (BMAT) at the time of diagnosis. The subject receives motor skill restoration therapy to improve the subject's impaired motor skills including physical therapy and strength training weekly for 6 months. Between 5 months and 6 months the subject shows less limited improvements in motor function as determined by the BMAT and is therefore deemed to be a therapy refractory subject. The clinician may determine the subject has a chronic TBI, and the lack of improvement in motor function may be because the critical window for relearning motor skills after a TBI is now closed, preventing the subject from regaining further motor function.

Once the subject has been determined to be refractory to therapy, the subject is administered between 50 and 250 μg of LSD orally once every 3 days for a period of 4 weeks. The subject is administered physical therapy daily and between 4 hours and 6 hours of being administered the pharmaceutically effective amount of LSD. After 4 weeks, the subject's motor function is assessed using the BMAT to identify any improvements in motor function.

### Example 2: Improving Motor Function in a Subject with an Acute Spinal Cord Injury

A subject suffering from an acute spinal cord injury (SCI) may be treated with 3,4-methylenedioxymethamphetamine (MDMA). A subject is diagnosed by a clinician with an SCI as a result of an external force using a physical exam and a CT scan. As a result of the SCI, the subject has impaired motor function in the lower extremities. In order to extend the critical window for relearning motor function, the subject is administered between 50 and 75 mg of MDMA within 1 month of sustaining the spinal cord injury. Within 1 hour of being administered MDMA, the subject is administered physical therapy to improve the subject's motor function. The subject is administered the pharmaceutical composition containing MDMA every 2 weeks for the period of 1 year or longer to prevent the critical window for relearning motor function from closing. The subject's motor function is assessed periodically by a clinician to identify improvements.

### Example 3: Improving Motor Function in a Subject Who has Suffered a Stroke

A subject who has experienced a stroke may be treated with ketamine. The subject is diagnosed by a clinician as having a central nervous system injury caused by a stroke using a physical exam and an MRI. As a result of the stroke, the subject has impaired speech. The subject receives physical therapy and speech therapy for 1 year, but no improvement is observed in the subject's ability to speak. The subject is determined to be therapy refractive and the critical window for relearning motor function is determined to be closed. The subject is administered between 20 mg and 200 mg of ketamine by intravenous infusion daily for a period of 1 week. The subject is administered speech therapy daily within 4 hours of being administered the pharmaceutical composition of ketamine. After two weeks, the subject's speech is assessed by a clinician to identify any improvements.

### Example 4: Treating Focal Diaschisis in a Subject Suffering from a Concussion

A subject sustains a concussion as a result of an external force to the head and is experiencing impairments in fine motor function. The subject is diagnosed by a clinician with focal diaschisis upon being administered a neurological exam and an MRI. The subject is administered an intravenous infusion of psilocybin or psilocin at a rate of 6 mg/hr within 7 days of receiving the concussion in order to extend the critical period of relearning motor functions in the subject. The subject is administered the pharmaceutical composition of psilocybin or psilocin every 2 weeks for a period of 4 months. The subject is administered physical therapy and occupational therapy within 6 hours of being administered the pharmaceutical composition of psilocybin or psilocin and daily for three days following being administered the pharmaceutical composition of psilocybin or psilocin. After 4 months of treatment the subject's fine motor skills are assessed to observe any improvements.

### Example 5: Treating Loss of Motor Function in a Subject Who has Suffered a Stroke Using MDMA

Subjects who have suffered a stroke within the past month are administered 75 mg of MDMA in a quiet, comfortable in-patient setting. All subjects are kept at the clinical site for at least 8 hours post-dosing for safety monitoring. A follow-up visit is conducted approximately 2 weeks, 6 weeks, and 12 weeks after the experimental session.

On the day of the procedure, the subject is taken to the designated session room. The procedure of events are described to the volunteer. Systolic and diastolic blood pressure, heart rate, and sublingual temperature are measured twice before ingestion of MDMA (at about 15 and 5 min prior to ingestion), and approximately every 20 min during the first hour after administration of MDMA, and thereafter about every 60 min until the end of the experiment. During the duration of the session, the monitors are vigilant for potential adverse psychological reactions. In the event of a significant adverse psychological reaction, interpersonal support is provided.

After administration of the 75 mg of MDMA, the subject encouraged to focus their attention inward by wearing eyeshades and listening to music through headphones. Monitors occasionally probe the subject's psychological well-being (e.g., ask the subject, “Would you like to describe where you find yourself?”) to ensure that the subject is not experiencing significant anxiety and is in need of support. The subject is free at any time to remove the eyeshades and/or music and reflect on the experience or engage in conversation with the monitors. If the subject becomes anxious during the course of MDMA action, the monitors provide strong personal support and reassurance, primarily including interacting with the subject in a comforting and reassuring manner. If the subject is behaving anxiously and a negative psychological reaction seems to be escalating, the monitors convey a solid sense of security and calm, while empathizing with what may be an incredibly intense and unpleasant experience. A physician is available during MDMA sessions should any untoward medical complications arise. Furthermore, medication for the treatment of acute hypertension (e.g., intravenous labetalol) is immediately available in the event that blood pressure exceeds pre-determined safety parameters.

After administration of the MDMA, the participant is invited to engage in the robotic therapy platform if the monitors deem the participant ready for such a task. The robotic therapy platform is already in the private session room ready for use. Video recordings of the subject are made as they perform upper extremity movements on the robotic therapy platform (see, Arac et al., 2019. Front Syst. Neurosci., 13, 20). The subject is asked to perform reaching movements. The subject is also subjected to Fugl-Meyer and ARAT tests right after the video recordings. These tests are scores based on ordinal scales as the subjects perform certain tasks. The video recordings take less than 10 min. Fugl-Meyer and ARAT tests each take ˜30 min to administer to the subject. From the videos, machine learning techniques to compare the movements of the subjects and identify different types of patterns are applied.

After the session, safety monitoring continues in the form of one or more post-session meetings (typically the next day) between the primary monitor and participant to ensure psychological stability and provide an opportunity for the volunteer to discuss thoughts or feelings from the session. As with any acute, intense positive or negative emotional experience, participants often feel the need for, and seem to benefit from, additional time for reflecting on the novel thoughts and feelings that may have arisen in the session. Given the potentially intense and unusual psychological nature of MDMA effects, the subject may have difficulty discussing the experience with others in her or his life. Because the monitors are present during the session when the MDMA effects are experienced and have knowledge of a broad range of reported phenomena during drug action, the subject may feel more comfortable discussing her or his experiences with the monitors than with others. This follow up contact also allows the assessment of any potentially persisting adverse effects, including perceptual abnormalities. More than one post-session meeting may be necessary if the subject is experiencing psychological difficulty concerning thoughts and feelings encountered during the session. A clinically trained psychologist or psychiatrist familiar with altered states of consciousness is available if a patient appears to have developed psychological difficulties stemming from MDMA administration.

Subjects are released into the care of a friend or family member, who has been appropriately oriented by the study personnel to be available to emotionally support the participant, but also to provide space (i.e., be in another room) if the subject feels the need to be alone.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of improving motor function in a therapy-refractory subject diagnosed with a central nervous system injury, the method comprising:
(i) administering to the subject a pharmaceutical composition comprising a psychedelic compound at least 3 months after sustaining the central nervous system injury, and
(ii) after step (i), administering a motor skill restoration therapy, thereby improving motor function in the therapy-refractory subject.

2. A method of improving motor function in a subject diagnosed with a central nervous system injury, the method comprising:
(i) administering to the subject a pharmaceutical composition comprising a psychedelic compound within 3 months of sustaining the central nervous system injury, and
(ii) after step (i), administering a motor skill restoration therapy, thereby improving motor function in the subject.

3. A method of treating focal diaschisis caused by a central nervous system injury in a subject, the method comprising:
(i) administering to the subject a pharmaceutical composition comprising a psychedelic compound, and
(ii) after step (i), administering a motor skill restoration therapy, thereby improving motor function in the subject.

4. The method of claim 3, wherein the subject suffers from crossed cerebellar diaschisis (CCD).

5. The method of claim 3 or 4, wherein the subject suffers from stroke, concussion, or chronic traumatic encephalopathy (CTE).

6. The method of any one of claims 1-5, wherein the motor skill restoration therapy is administered for an additional 2 to 4 weeks after the psychedelic compound is no longer being administered to the subject.

7. The method of any one of claims 1-6, wherein the motor skill restoration therapy is administered within 24 hours of the psychedelic compound.

8. The method of claim 7, wherein the motor skill restoration therapy is administered within 6 hours of the psychedelic compound.

9. The method of claim 8, wherein the motor skill restoration therapy is administered within 1 hour of the psychedelic compound.

10. The method of any one of claims 1-9, wherein the motor skill restoration therapy comprises a rehabilitation program and/or a therapeutic compound.

11. The method of claim 10, wherein the motor skill restoration therapy comprises a rehabilitation program.

12. The method of claim 11, wherein the rehabilitation program comprises physical therapy, occupational therapy, or speech therapy.

13. The method of claim 11 or claim 12, wherein the rehabilitation program comprises a robot assisted therapy.

14. The method of claim 13, wherein the robot assisted therapy is an end-effector type robotic device therapy.

15. The method of claim 13, wherein the robot assisted therapy is an exoskeleton-type robotic device therapy.

16. The method of any one of claims 1-15, wherein the central nervous system injury results from a stroke.

17. The method of any one of claims 1-15, wherein the central nervous system injury is a traumatic brain injury.

18. The method of any one of claims 1-15, wherein the central nervous system injury is a spinal cord injury.

19. The method of claim 18, wherein the subject is administered an alpha adrenergic drug.

20. The method of any one of claims 1-19, wherein the motor function is a fine or a gross motor function.

21. The method of claim 20, wherein the motor function is a fine motor function.

22. The method of claim 21, wherein the fine motor function is a speech.

23. The method of any one of claims 1-22, wherein the psychedelic compound is administered every 3 to 4 days.

24. The method of any one of claims 1-22, wherein the psychedelic compound is administered every 1 to 2 weeks.

25. The method of any one of claims 1-22, wherein the psychedelic compound is administered every 3 to 4 weeks.

26. The method of any one of claims 1-25, wherein the psychedelic compound is a 5-HT_{2A} receptor agonist.

27. The method of claim 26, wherein the 5-HT_{2A} receptor agonist is lysergic acid diethylamide.

28. The method of claim 27, wherein said pharmaceutical composition is a unit dosage form comprising from 5 to 250 μg of lysergic acid diethylamide or a pharmaceutically acceptable salt thereof.

29. The method of claim 28, wherein the unit dosage is an oral dose.

30. The method of claim 28 or 29, wherein the unit dosage form comprises from 5 to 250 μg of lysergic acid diethylamide or a pharmaceutically acceptable salt thereof.

31. The method of claim 30, wherein the unit dosage form comprises from 5 to 30 μg of lysergic acid diethylamide or a pharmaceutically acceptable salt thereof.

32. The method of claim 31, wherein said pharmaceutical composition is a unit dosage form comprising 10±5 μg of lysergic acid diethylamide or a pharmaceutically acceptable salt thereof.

33. The method of claim 26, wherein the 5-HT_{2A} receptor agonist is psilocybin or psilocin.

34. The method of claim 33, wherein said pharmaceutical composition is a unit dosage form comprising from 1 to 40 mg of psilocybin or psilocin or a pharmaceutically acceptable salt thereof

35. The method of claim 34, wherein the unit dosage form comprises from 1 to 10 mg of psilocybin or psilocin or a pharmaceutically acceptable salt thereof.

36. The method of claim 34, wherein the unit dosage form comprises from 5 to 40 mg of psilocybin or psilocin or a pharmaceutically acceptable salt thereof.

37. The method of any one of claims 1-36, wherein the psychedelic compound or pharmaceutically acceptable salt thereof is administered orally, intravenously, by inhalation, by nebulization, by aerosolization, intranasally, intratracheally, intrabronchially, subcutaneously, or intramuscularly.

38. The method of claim 37, wherein the psychedelic compound is administered intravenously.

39. The method of claim 37, wherein the psychedelic compound is administered orally.

40. A method of improving motor function in a therapy-refractory subject diagnosed with a central nervous system injury, the method comprising:
(i) administering to the subject a pharmaceutical composition comprising an empathogenic compound at least 3 months after sustaining the central nervous system injury, and
(ii) after step (i), administering a motor skill restoration therapy, thereby improving motor function in the therapy-refractory subject.

41. A method of improving motor function in a subject diagnosed with a central nervous system injury, the method comprising:
(i) administering to the subject a pharmaceutical composition comprising an empathogenic compound within 3 months of sustaining the central nervous system injury, and
(ii) after step (i), administering a motor skill restoration therapy, thereby improving motor function in the subject.

42. A method of treating focal diaschisis caused by a central nervous system injury in a subject, the method comprising:
(i) administering to the subject a pharmaceutical composition comprising an empathogenic compound, and
(ii) after step (i), administering a motor skill restoration therapy, thereby improving motor function in the subject.

43. The method of claim 42, wherein the subject suffers from CCD.

44. The method of claim 42 or 43, wherein the subject suffers from stroke, concussion, or CTE.

45. The method of any one of claims 40-44, wherein the motor skill restoration therapy is administered for an additional 2 to 4 weeks after the empathogenic compound is no longer being administered to the subject.

46. The method of any one of claims 40-45, wherein the motor skill restoration therapy is administered within 24 hours of the empathogenic compound.

47. The method of claim 46, wherein the motor skill restoration therapy is administered within 6 hours of the empathogenic compound.

48. The method of claim 47, wherein the motor skill restoration therapy is administered within 1 hour of the empathogenic compound.

49. The method of any one of claims 40-48, wherein the motor skill restoration therapy comprises a rehabilitation program and/or a therapeutic compound.

50. The method of claim 49, wherein the motor skill restoration therapy comprises a rehabilitation program.

51. The method of claim 50, wherein the rehabilitation program comprises physical therapy, occupational therapy, or speech therapy.

52. The method of claim 51, wherein the rehabilitation program comprises a robot assisted therapy.

53. The method of claim 52, wherein the robot assisted therapy is an end-effector type robotic device therapy.

54. The method of claim 52, wherein the robot assisted therapy is an exoskeleton-type robotic device therapy.

55. The method of any one of claims 40-54, wherein the central nervous system injury results from a stroke.

56. The method of any one of claims 40-54, wherein the central nervous system injury is a traumatic brain injury.

57. The method of any one of claims 40-54, wherein the central nervous system injury is a spinal cord injury.

58. The method of claim 57, wherein the subject is administered an alpha adrenergic drug.

59. The method of any one of claims 40-58, wherein the motor function is a fine or a gross motor function.

60. The method of claim 59, wherein the motor function is a fine motor function.

61. The method of claim 60, wherein the fine motor function is speech.

62. The method of any one of claims 40-61, wherein the empathogenic compound is administered every 3 to 4 days.

63. The method of any one of claims 40-61, wherein the empathogenic compound is administered every 1 to 2 weeks.

64. The method of any one of claims 40-61, wherein the empathogenic compound is administered every 3 to 4 weeks.

65. The method of any one of claims 40-64, wherein the empathogenic compound is 3,4-methylenedioxymethamphetamine (MDMA).

66. The method of claim 65, wherein said pharmaceutical composition is a unit dosage form comprising from 50 to 150 mg of 3,4-methylenedioxymethamphetamine or a pharmaceutically acceptable salt thereof.

67. The method of claim 66, wherein the unit dosage form is an oral dose.

68. The method of claim 66 or claim 67, wherein the unit dosage comprises from 50 to 75 mg of 3,4-methylenedioxymethamphetamine or a pharmaceutically acceptable salt thereof.

69. The method of claim 66 or claim 67, wherein the unit dosage comprises from 75 to 150 mg of 3,4-methylenedioxymethamphetamine or a pharmaceutically acceptable salt thereof.

70. The method of any one of claims 65-68, wherein the unit dosage comprises 75 mg of 3,4-methylenedioxymethamphetamine or a pharmaceutically acceptable salt thereof.

71. The method of any one of claims 40-69, wherein the empathogenic compound or pharmaceutically acceptable salt thereof is administered is orally, intravenously, by inhalation, by nebulization, by aerosolization, intranasally, intratracheally, intrabronchially, subcutaneously, or intramuscularly

72. A method of improving motor function in a therapy-refractory subject diagnosed with a central nervous system injury, the method comprising:
(i) administering to the subject a pharmaceutical composition comprising a dissociative compound at least 3 months after sustaining the central nervous system injury, and
(ii) after step (i), administering a motor skill restoration therapy, thereby improving motor function in the therapy-refractory subject.

73. A method of improving motor function in a subject diagnosed with a central nervous system injury, the method comprising:
(i) administering to the subject a pharmaceutical composition comprising a dissociative compound within 3 months of sustaining the central nervous system injury, and
(ii) after step (i), administering a motor skill restoration therapy, thereby improving motor function in the subject.

74. A method of treating focal diaschisis caused by a central nervous system injury in a subject, the method comprising:
(i) administering to the subject a pharmaceutical composition comprising a dissociative compound, and
(ii) after step (i), administering a motor skill restoration therapy, thereby improving motor function in the subject.

75. The method of claim 74, wherein the subject suffers from CCD.

76. The method of claim 74 or 75, wherein the subject suffers from stroke, concussion, or CTE.

77. The method of any one of claims 72-76, wherein the motor skill restoration therapy is administered for an additional 2 to 4 weeks after the dissociative compound is no longer being administered to the subject.

78. The method of any one of claims 72-77, wherein the motor skill restoration therapy is administered within 24 hours of the dissociative compound.

79. The method of claim 78, wherein the motor skill restoration therapy is administered within 6 hours of the dissociative compound.

80. The method of claim 79, wherein the motor skill restoration therapy is administered within 1 hour of the dissociative compound.

81. The method of any one of claims 72-80, wherein the motor skill restoration therapy comprises a rehabilitation program and/or a therapeutic compound.

82. The method of claim 81, wherein the motor skill restoration therapy comprises a rehabilitation program.

83. The method of claim 82, wherein the rehabilitation program comprises physical therapy, occupational therapy, or speech therapy.

84. The method of claim 82, wherein the rehabilitation program comprises a robot assisted therapy.

85. The method of claim 84, wherein the robot assisted therapy is an end-effector type robotic device therapy.

86. The method of claim 84, wherein the robot assisted therapy is an exoskeleton-type robotic device therapy.

87. The method of any one of claims 72-86 wherein the central nervous system injury results from a stroke.

88. The method of any one of claims 72-86, wherein the central nervous system injury is a traumatic brain injury.

89. The method of any one of claims 72-86, wherein the central nervous system injury a spinal injury.

90. The method of claim 89, wherein the subject is administered an alpha adrenergic drug.

91. The method of any one of claims 72-90, wherein the motor function is a fine or a gross motor function.

92. The method of claim 91, wherein the motor function is a fine motor function.

93. The method of claim 92, wherein the fine motor function is speech.

94. The method of any one of claims 72-93, wherein the dissociative compound is administered every 3 to 4 days.

95. The method of any one of claims 72-93, wherein the dissociative compound is administered every 1 to 2 weeks.

96. The method of any one of claims 72-93, wherein the dissociative compound is administered every 3 to 4 weeks.

97. The method of any one of claims 72-96, wherein the dissociative compound is ketamine.

98. The method of claim 97, wherein said pharmaceutical composition is a unit dosage form comprising from 50 to 200 mg of ketamine or a pharmaceutically acceptable salt thereof.

99. The method of claim 98, wherein the unit dosage form is an intravenous dose.

100. The method of claim 98 or claim 99, wherein said pharmaceutical composition is a unit dosage form comprising 100±10 mg of ketamine or a pharmaceutically acceptable salt thereof.

101. The method of any one of claims 72-100, wherein the dissociative compound or pharmaceutically acceptable salt thereof is administered is orally, intravenously, by inhalation, by nebulization, by aerosolization, intranasally, intratracheally, intrabronchially, subcutaneously, or intramuscularly

102. The method of any one of claims 1-101, wherein the subject is a human.
